(19) United States
(12) Patent Application Publication
(10) Pub. No.: US 2004/0034042 A1
(43) Pub. Date: Feb. 19, 2004

(54) Preservative composition

(76) Inventors: Masao Tsuji, Osaka-shi (JP); Tadashi Seto, Osaka-shi (JP); Yasuko Mori, Osaka-shi (JP); Yuka Kiyobayashi, Osaka-shi (JP); Tetsuo Koike, Osaka-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/421,977

(22) Filed: Apr. 23, 2003

(30) Foreign Application Priority Data
Aug. 14, 2002 (JP) 2002-236479

(51) Int. Cl. A61K 31/522

(52) U.S. Cl. 514/263.31

(57) ABSTRACT

The present invention provides, as a composition that is highly safe and superior in preservative properties, comprising (a) a xanthine, (b) a buffer and (c) at least one member selected from sorbic acid, EDTA, and salts thereof. This composition has superior preservative properties so that it inhibits the generation and proliferation of microorganisms even when stored for a long period of time. Furthermore, the present invention provides a method for enhancing the preservative properties of sorbic acid, EDTA, and salts thereof, which are known to have preservative properties, and the preservative properties of compositions containing these ingredients, and provides a method for producing a composition with superior preservative effectiveness.

### TECHNICAL FIELD

The present invention is directed to a composition having preservative properties. More specifically, the present invention relates to a composition that is highly safe and of superior preservative effectiveness. Moreover, the present invention relates to a method for enhancing the preservative properties of sorbic acid, ethylenediaminetetraacetic acid, and their salts, which are known to have preservative properties.

### BACKGROUND OF THE INVENTION

Ophthalmic solutions, ophthalmic ointments and like pharmaceuticals are generally produced as germ-free compositions during their production processes. However, once the seal of their containers is opened, but prior to these pharmaceuticals are completely used up over one or more months, these containers are continuously used by opening or closing their cover, and therefore these pharmaceuticals are exposed to the risk of being contaminated by the microorganisms existing in the environment or the human body. Further, compositions such as food products, cosmetics, quasi-medical products, etc., are not necessarily produced as perfectly germ-free compositions in their production processes. In this regard, upon production of these compositions, a variety of treatments or techniques are necessary to prevent microorganism contamination that may occur after containers are opened, or to prevent microorganism proliferation in these compositions by increasing their preservative efficacy.

Accordingly, various preservatives are usually used in compositions such as food products, cosmetics, pharmaceuticals, quasi-medical products and the like to maintain the preservative efficacy of these products. For example, in ophthalmic formulations, contact lens-care solutions and like aqueous ophthalmic formulations, benzalkonium chloride, benzethonium chloride, chlorhexidine gluconate, chlorobutanol, p-amino benzoate, alkyldiaminoethylglycine hydrochloride, sorbic acid and their salts, etc., are used alone or in combination as preservatives. Among these preservative compounds, sorbic acid and its salts are widely used as preservatives for food products and contact lens-care solution due to the fact that they are safer than benzalkonium chloride, benzethonium chloride and like cationic surfactant preservatives and due to lesser problems with adosorption on contact lenses. Further, recently, sorbic acid and its salts have been frequently used as preservatives for cosmetics, quasi-medical products, pharmaceuticals and a wide variety of like products in addition to food products and contact lens-care solutions.

Needless to say, from the viewpoint of achieving more safety, however, it is desirable to reduce the amount of these preservatives used despite being considered relatively safe.

Pharmaceuticals (e.g., ophthalmic formulations) and cosmetics prepared in unit-dose form (tightly sealed, germ-free preparations provided in an amount suitable for single use) that do not contain preservatives are now known. The production cost of the preparations in unit dose form is high, and therefore, the preparations are unavailable to consumers at low price.

Although sorbic acid and its salts are very safe, the preservative properties, however, are weaker than that of cationic surfactant preservatives (e.g., benzalkonium chloride, benzethonium chloride, etc.) and other preservatives. In this respect, various research efforts have been made to enhance the preservative properties of sorbic acid and its salts. For example, Japanese Unexamined Patent Publication No. 292793/1999 discloses the enhancement of the preservative properties of sorbic acid by mixing an aromatic, sodium edetate, etc., with sorbic acid. The improved preservative properties are however insufficient.

It is also known that caffeine exhibits antibacterial activity to Rhizopus, Penicillium and like fungi and inhibits the production of aflatoxin by A. parasiticus (Journal of Food Protection, Vol. 45, No. 10, pp. 953-963, 1982); these effects are also insufficient.

### DISCLOSURE OF THE INVENTION

An object of the present invention is to overcome the prior art problems described above. Specifically, an object of the invention is to provide a composition (preservative composition) that is highly safe and has a superior preservative property. Another object of the invention is to provide a composition especially suitable for application to the skin or mucous membranes as a preservative composition. Yet another object of the invention is to provide a method for enhancing preservative properties of known preservatives such as sorbic acid, ethylenediaminetetraacetic acid, and their salts, and for enhancing preservative properties of preservative compositions containing them.

The inventors conducted extensive research to achieve the above objectives and found that by mixing a xanthine, as typically represented by caffeine, and a buffer with sorbic acid, ethylenediaminetetraacetic acid, or their salts, which have been known to have preservative properties, the preservative properties of these compounds are synergistically enhanced. The inventors confirmed that the composition thus obtained is useful as a composition having high preservative properties that can inhibit the generation and proliferation of microorganisms even when stored for a long period of time. The present invention has been developed based on the above findings.

Particularly, the present invention provides the compositions described in Items (1) to (12) below:

(1) A composition comprising:

(a) a compound represented by Formula (I):

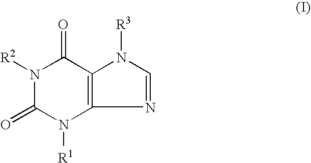

wherein R¹, R² and R³ are the same or different, and individually represent hydrogen or optionally substituted alkyl, or a salt of the compound;

(b) a buffer, and

(c) at least one member selected from sorbic acid, ethylenediaminetetraacetic acid, and salts thereof.

(2) A composition according to Item (1), wherein the composition is an aqueous composition.

(3) A composition according to Items (1) or (2), wherein the composition is suitable for application to the skin or mucous membranes.

(4) A composition according to any of Items (1) to (3), wherein the composition is an ophthalmic composition or an oral composition.

(5) A composition according to any of Items (1) to (4), wherein the buffer is a borate buffer, phosphate buffer, carbonate buffer, citrate buffer or acetate buffer.

(6) A composition according to any of Items (1) to (4), wherein the buffer is a borate buffer or phosphate buffer.

(7) A composition according to any of Items (1) to (6), wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

(8) A composition according to any of Items (1) to (6), wherein Compound (I) is at least one compound selected from caffeine, pentoxifylline, theophylline, diprophylline, theobromine and proxyphylline.

(9) A composition according to any of Items (1) to (8), wherein Compound (I) is at least one compound selected from caffeine-sodium benzoate, caffeine citrate, aminophylline, ambuphylline, theobromine calcium salicylate, theobromine sodium salicylate and theobromine sodium acetate.

(10) A composition according to any of Items (1) to (9) wherein the composition comprises (a) Compound (I) or a salt thereof in a concentration of 0.0001 to 10 w/v %, (b) the buffer in a concentration of 0.0001 to 10 w/v %, and (c) the sorbic acid or a salt thereof in a concentration of 0.00005 to 10 w/v %, or the ethylenediaminetetraacetic acid or a salt thereof in a concentration of 0.001 to 1 w/v %.

(11) A composition according to any of Items (1) to (10), wherein the composition comprises (b) the buffer in a proportion of 0.1 to 500 parts by weight and (c) the sorbic acid or a salt thereof in a total proportion of 0.002 to 100 parts by weight, or the ethylenediaminetetraacetic acid or a salt thereof in a total proportion of 0.001 to 1,000 parts by weight, per part by weight of the total amount of (a) Compound (I) or a salt thereof.

(12) A composition according to any of Items (1) to (11), wherein the composition is an ophthalmic solution, collyrium, ophthalmic ointment, contact lens-wearing solution, or contact lens-care formulation.

In addition, the present invention provides methods as described in Items (13) to (20) below for synergistically enhancing the preservative properties of Compound (I) or a salt thereof, and the preservative properties of sorbic acid, ethylenediaminetetraacetic acid, or a salt thereof:

(13) A method for synergistically enhancing the preservative properties of the following ingredient (a) and (c), comprising employing in combination:

(a) a compound represented by Formula (I):

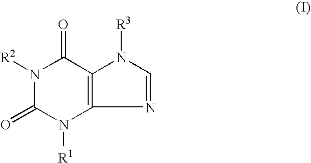

wherein R¹, R² and R³ are as defined above, or a salt of the compound;

(b) a buffer; and

(c) at least one member selected from sorbic acid, ethylenediaminetetraacetic acid, and salts thereof.

(14) A method according to Item (13), wherein the buffer is a borate buffer, phosphate buffer, carbonate buffer, citrate buffer or acetate buffer.

(15) A method according to Item (13), wherein the buffer is a phosphate buffer or borate buffer.

(16) A method according to any of Items (13) to (15), wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

(17) A method according to any of Items (13) to (15), wherein Compound (I) is at least one compound selected from caffeine, pentoxifylline, theophylline, diprophylline, theobromine and proxyphylline.

(18) A method according to any of Items (13) to (15), wherein Compound (I) is at least one compound selected from caffeine-sodium benzoate, caffeine citrate, aminophylline, ambuphylline, theobromine calcium salicylate, theobromine sodium salicylate and theobromine sodium acetate.

(19) A method according to any of Items (13) to (18), wherein (a) Compound (I) or a salt thereof is used in a proportion of 0.01 to 500 parts by weight per part by weight of the total amount of (c) the sorbic acid or a salt thereof, and (b) the buffer is used in a proportion of 0.1 to 500 parts by weight per part by weight of the total amount of (a) Compound (I) or a salt thereof.

(20) A method according to any of Items (13) to (18), wherein (a) Compound (I) or a salt thereof is used in a proportion of 0.001 to 1,000 parts by weight per part by weight of the total amount of (c) the ethylenediaminetetraacetic acid or a salt thereof, and (b) the buffer is used in a proportion of 0.1 to 500 parts by weight per part by weight of the total amount of (a) Compound (I) or a salt thereof.

Moreover, the present invention provides methods as described in Items (21) to (28) below for enhancing the preservative properties of sorbic acid, ethylenediaminetetraacetic acid, or a salt thereof:

(21) A method for enhancing the preservative properties of the following ingredient (c), comprising employing:

(a) a compound represented by following Formula (I):

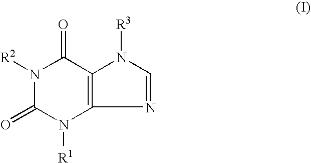

wherein R¹, R² and R³ are as defined above, or a salt of the compound and

(b) a buffer in combination with

(c) at least one member selected from sorbic acid, ethylenediaminetetraacetic acid, and salts thereof.

(22) A method according to Item (21), wherein the buffer is a borate buffer, phosphate buffer, carbonate buffer, citrate buffer or acetate buffer.

(23) A method according to Item (21), wherein the buffer is a phosphate buffer or borate buffer.

(24) A method according to any of Items (21) to (23), wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

(25) A method according to any of Items (21) to (23), wherein Compound (I) is at least one compound selected from caffeine, pentoxifylline, theophylline, diprophylline, theobromine and proxyphylline.

(26) A method according to any of Items (21) to (23), wherein Compound (I) is at least one compound selected from caffeine-sodium benzoate, caffeine citrate, aminophylline, ambuphylline, theobromine calcium salicylate, theobromine sodium salicylate and theobromine sodium acetate.

(27) A method according to any of Items (21) to (26), wherein (a) Compound (I) or a salt thereof is used in a proportion of 0.01 to 500 parts by weight per part by weight of the total amount of (c) the sorbic acid or a salt thereof, and (b) the buffer is used in a proportion of 0.1 to 500 parts by weight per part by weight of the total amount of (a) Compound (I) or a salt thereof. (28) A method according to any of Items (21) to (26), wherein (a) Compound (I) or a salt thereof is used in a proportion of 0.001 to 1,000 parts by weight per part by weight of the total amount of (c) the ethylenediaminetetraacetic acid or a salt thereof, and (b) the buffer is used in a proportion of 0.1 to 500 parts by weight per part by weight of the total amount of (a) Compound (I) or a salt thereof.

Further, the present invention provides methods as described in Items (29) to (38) below for enhancing the preservative properties of a composition comprising sorbic acid, ethylenediaminetetraacetic acid, or a salt thereof:

(29) A method for enhancing the preservative properties of a composition comprising at least one member selected from sorbic acid, ethylenediaminetetraacetic acid, and salts thereof, the method comprising employing:

the above-mentioned composition, in combination with a compound represented by Formula (I):

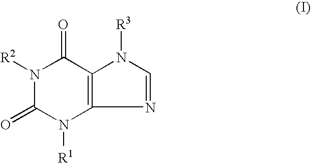

wherein R¹, R² and R³ are as defined above, or a salt of the compound; and a buffer.

(30) A method according to Item (29), wherein the buffer is a borate buffer, phosphate buffer, carbonate buffer, citrate buffer or acetate buffer.

(31) A method according to Item (29), wherein the buffer is a phosphate buffer or borate buffer.

(32) A method according to any of Items (29) to (31), wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

(33) A method according to any of Items (29) to (31), wherein Compound (I) is at least one compound selected from caffeine, pentoxifylline, theophylline, diprophylline, theobromine and proxyphylline.

(34) A method according to any of Items (29) to (31), wherein Compound (I) is at least one compound selected from caffeine-sodium benzoate, caffeine citrate, aminophylline, ambuphylline, theobromine calcium salicylate, theobromine sodium salicylate and theobromine sodium acetate.

(35) A method according to any of Items (29) to (34), wherein a final formulation obtained by mixing Compound (I) or a salt thereof, and the buffer with the composition containing at least one member selected from sorbic acid, ethylenediaminetetraacetic acid and salts thereof comprises the sorbic acid or a salt thereof in a concentration of 0.00005 to 10 w/v %, or the. ethylenediaminetetraacetic acid or a salt thereof in a concentration of 0.001 to 1 w/v %.

(36) A method according to Items (29) to (35), wherein the composition containing at least one member selected from sorbic acid, ethylenediaminetetraacetic acid, and salts thereof comprises the ethylenediaminetetraacetic acid or a salt thereof in a proportion of 0.001 to 100 parts by weight per part by weight of the total amount of the sorbic acid or a salt thereof.

(37) A method according to any of Items (29) to (36), wherein (a) Compound (I) or a salt thereof is used in a proportion of 0.01 to 500 parts by weight per part by weight of the total amount of (c) the sorbic acid or a salt thereof, and (b) the buffer is used in a proportion of 0.1 to 500 parts by weight per part by weight of the total amount of (a) Compound (I) or a salt thereof.

(38) A method according to any of Items (29) to (36), wherein (a) Compound (I) or a salt thereof is used in a proportion of 0.001 to 1,000 parts by weight per part by weight of the total amount of (c) the ethylenediaminetetraacetic acid or a salt thereof, and (b) the buffer is used in a proportion of 0.1 to 500 parts by weight per part by weight of the total amount of (a) Compound (I) or a salt thereof.

From another perspective, the methods of Items (29) to (38) set forth above can be rewritten as follows:

(39) A method for preparing a preservative composition having enhanced preservative properties, the method comprising:

mixing a compound represented by Formula (I):

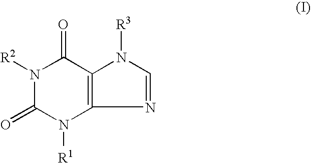

wherein R¹, R² and R³ are as defined above, or a salt of the compound; and a buffer with a composition comprising at least one member selected from the group consisting of sorbic acid, ethylenediaminetetraacetic acid, and salts thereof.

Furthermore, the present invention provides methods as described in Items (40) to (49) below for enhancing the preservative properties of a composition comprising sorbic acid, ethylenediaminetetraacetic acid, or a salt thereof, and a buffer:

(40) A method for enhancing the preservative properties of a composition comprising a buffer and at least one member selected from sorbic acid, ethylenediaminetetraacetic acid, and salts thereof, the method comprising employing in combination:

the above-mentioned composition, and a compound represented by Formula (I):

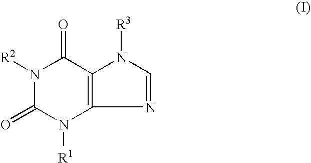

wherein R¹, R² and R³ are as defined above, or a salt of the compound.

(41) A method according to Item (40), wherein the buffer is a borate buffer, phosphate buffer, carbonate buffer, citrate buffer or acetate buffer.

(42) A method according to Item (40), wherein the buffer is a phosphate-buffer or borate buffer.

(43) A method according to any of Items (40) to (42), wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

(44) A method according to any of Items (40) to (42), wherein Compound (I) is at least one compound selected from caffeine, pentoxifylline, theophylline, diprophylline, theobromine and proxyphylline.

(45) A method according to any of Items (40) to (42), wherein Compound (I) is at least one compound selected from caffeine-sodium benzoate, caffeine citrate, aminophylline, ambuphylline, theobromine calcium salicylate, theobromine sodium salicylate and theobromine sodium acetate.

(46) A method according to any of Items (40) to (45), wherein a final formulation obtained by mixing Compound (I) or a salt thereof with the composition containing a buffer and at least one member selected from sorbic acid, ethylenediaminetetraacetic acid, and salts thereof comprises the sorbic acid or a salt thereof in a concentration of 0.00005 to 10 w/v % or the ethylenediaminetetraacetic acid or a salt thereof in a concentration of 0.001 to 1 w/v %, and the buffer in a concentration of 0.0001 to 10 w/v %.

(47) A method according to Items (40) to (46), wherein the composition comprising a buffer and at least one member selected from sorbic acid, ethylenediaminetetraacetic acid, and salts thereof comprises the ethylenediaminetetraacetic acid or a salt thereof in a proportion of 0.001 to 100 parts by weight per part by weight of the total amount of the sorbic acid or a salt thereof.

(48) A method according to any of Items (40) to (47), wherein (a) Compound (I) or a salt thereof is used in a proportion of 0.01 to 500 parts by weight per part by weight of the total amount of (c) the sorbic acid or a salt thereof; or (a) Compound (I) or a salt thereof is used in a proportion of 0.2 to 1,000 parts by weight based on 100 parts by weight of the buffer.

(49) A method according to any of Items (40) to (47), wherein (a) Compound (I) or a salt thereof is used in a proportion of 0.001 to 1,000 parts by weight per part by weight of the total amount of (c) the ethylenediaminetetraacetic acid or a salt thereof; or (a) Compound (I) or a salt thereof is used in a proportion of 0.2 to 1,000 parts by weight based on 100 parts by weight of the buffer.

From another perspective, the methods of Items (40) to (49) set forth above can be rewritten as follows:

(50) A method for preparing a preservative composition having enhanced preservative properties, the method comprising mixing a compound represented by Formula (I):

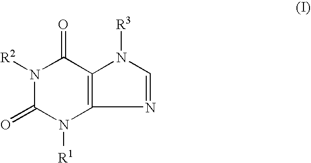

wherein R¹, R² and R³ are as defined above, or a salt of the compound; with a composition containing a buffer and at least one member selected from sorbic acid, ethylenediaminetetraacetic acid, and salts thereof.

The present invention also provides methods as described in Items (51) to (58) below for enhancing the preservative properties of Compound (I) or a salt thereof:

(51) A method for enhancing the preservative properties of Compound (I) or a salt thereof, the method comprising employing:

(a) a compound represented by Formula (I):

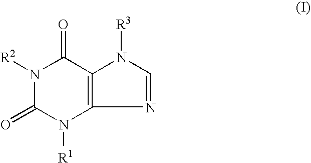

wherein R¹, R² and R³ are as defined above, or a salt of the compound; in combination with

(b) a buffer and

(c) one member selected from sorbic acid, ethylenediaminetetraacetic acid, and salts thereof.

(52) A method according to Item (51), wherein the buffer is a borate buffer, phosphate buffer, carbonate buffer, citrate buffer or acetate buffer.

(53) A method according to Item (51), wherein the buffer is a phosphate buffer or borate buffer.

(54) A method according to any of Items (51) to (53), wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

(55) A method according to any of Items (51) to (53), wherein Compound (I) is at least one compound selected from caffeine, pentoxifylline, theophylline, diprophylline, theobromine and proxyphylline.

(56) A method according to any of Items (51) to (53), wherein Compound (I) is at least one compound selected from caffeine-sodium benzoate, caffeine citrate, aminophylline, ambuphylline, theobromine calcium salicylate, theobromine sodium salicylate and theobromine sodium acetate.

(57) A method according to any of Items (51) to (56), wherein (b) the buffer is used in a proportion of 0.1 to 500 parts by weight and (c) the sorbic acid or a salt thereof is used in a total proportion of 0.002 to 100 parts by weight per part by weight of the total amount of (a) Compound (I) or a salt thereof.

(58) A method according to any of Items (51) to (56), wherein (b) the buffer is used in a proportion of 0.1 to 500 parts by weight and (c) the ethylenediaminetetraacetic acid or a salt thereof is used in a total proportion of 0.001 to 1,000 parts by weight per part by weight of the total amount of (a) Compound (I) or a salt thereof.

In addition, the present invention provides methods as described in Items (59) to (67) below for enhancing the preservative properties of a composition comprising Compound (I) or a salt thereof:

(59) A method for enhancing the preservative properties of a composition containing a compound represented by Formula (I):

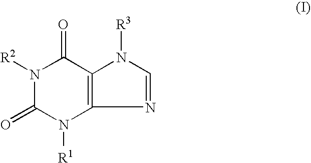

wherein R¹, R² and R³ are as defined above, or a salt of the compound, the method comprising employing: the above-mentioned composition in combination with a buffer and at least one member selected from sorbic acid, ethylenediaminetetraacetic acid, and salts thereof.

(60) A method according to Item (59), wherein the buffer is a borate buffer, phosphate buffer, carbonate buffer, citrate buffer or acetate buffer.

(61) A method according to Item (59), wherein the buffer is a phosphate buffer or borate buffer.

(62) A method according to any of Items (59) to (61), wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

(63) A method according to any of Items (59) to (61), wherein Compound (I) is at least one compound selected from caffeine, pentoxifylline, theophylline, diprophylline, theobromine and proxyphylline.

(64) A method according to any of Items (59) to (61), wherein Compound (I) is at least one compound selected from caffeine-sodium benzoate, caffeine citrate, aminophylline, ambuphylline, theobromine calcium salicylate, theobromine sodium salicylate and theobromine sodium acetate.

(65) A method according to any of Items (59) to (64), wherein a final formulation obtained by mixing the buffer and at least one member selected from sorbic acid, ethylenediaminetetraacetic acid, and salts thereof with the composition containing Compound (I) or a salt thereof comprises Compound (I) or a salt thereof in a concentration of 0.0001 to 10 w/v %.

(66) A method according to any of Items (59) to (65), wherein the buffer is used in a proportion of 0.1 to 500 parts by weight, and the sorbic acid or a salt thereof is used in a total proportion of 0.002 to 100 parts by weight per part by weight of the total amount of Compound (I) or a salt thereof.

(67) A method according to any of Items (59) to (65), wherein the buffer is used in a proportion of 0.1 to 500 parts by weight, and the ethylenediaminetetraacetic acid or a salt thereof is used in a total proportion of 0.001 to 1,000 parts by weight per part by weight of the total amount of Compound (I) or a salt thereof.

From another perspective, the methods of Items (59) to (67) set forth above can be rewritten as follows:

(68) A method for preparing a preservative composition having enhanced preservative properties, the method comprising mixing a buffer and at least one member selected from sorbic acid, ethylenediaminetetraacetic acid, and salts thereof with a composition comprising a compound represented by Formula (I):

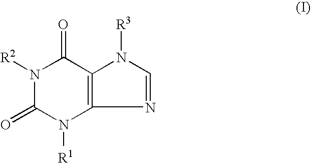

wherein R¹, R² and R³ are as defined above, or a salt of the compound.

Furthermore, the present invention provides methods as described in Items (69) to (79) below for enhancing the preservative properties of a composition comprising Compound (I) or a salt thereof, and a buffer.

(69) A method for enhancing the preservative properties of a composition comprising a buffer and a compound represented by Formula (I):

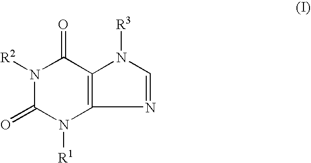

wherein R¹, R² and R³ are as defined above, or a salt the compound, the method comprising employing in combination at least one member selected from sorbic acid, ethylenediaminetetraacetic acid, and salts thereof; and the above-mentioned composition.

(70) A method according to Item (69), wherein the buffer is a borate buffer, phosphate buffer, carbonate buffer, citrate buffer or acetate buffer.

(71) A method according to Item (69), wherein the buffer is a phosphate buffer or borate buffer.

(72) A method according to any of Items (69) to (71), wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

(73) A method according to any of Items (69) to (71), wherein Compound (I) is at least one compound selected from caffeine, pentoxifylline, theophylline, diprophylline, theobromine and proxyphylline.

(74) A method according to any of Items (69) to (71), wherein Compound (I) is at least one compound selected from caffeine-sodium benzoate, caffeine citrate, aminophylline, ambuphylline, theobromine calcium salicylate, theobromine sodium salicylate and theobromine sodium acetate.

(75) A method according to any of Items (69) to (71), wherein a final formulation obtained by mixing at least one member selected from sorbic acid, ethylenediaminetetraacetic acid, and salts thereof with the composition containing the buffer and Compound (I) or a salt thereof comprises Compound (I) or a salt thereof in a concentration of 0.0001 to 10 w/v % and the buffer in a concentration of 0.0001 to 10 w/v %.

(76) A method according to any of Items (69) to (75), wherein the sorbic acid or a salt thereof is used in a total proportion of 0.002 to 100 parts by weight per part by weight of the total amount of Compound (I) or a salt thereof.

(77) A method according to any of Items (69) to (75), wherein the ethylenediaminetetraacetic acid or a salt thereof is used in a total proportion of 0.001 to 1,000 parts by weight per part by weight of the total amount of Compound (I) or a salt thereof.

From another perspective, the methods of Items (69) to (77) set forth above can be rewritten as follows:

(78) A method for preparing a preservative composition having enhanced preservative properties, the method comprising mixing at least one member selected from sorbic acid, ethylenediaminetetraacetic acid, and salts thereof with a composition containing a buffer and a compound represented by following Formula (I):

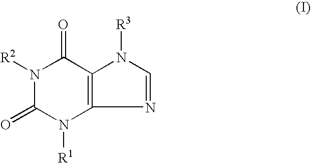

wherein R¹, R² and R³ are as defined above, or a salt of the compound.

The symbol “%” refers herein to “w/v %” unless specified otherwise. The term “contact lenses” is used herein to include any contact lenses regardless of type such as soft contact lenses, hard contact lenses, oxygen-permeable contact lenses, etc.

### BEST MODE FOR CARRYING OUT THE INVENTION

(1) Composition

The composition of the present invention comprises (a) a compound represented by Formula (I):

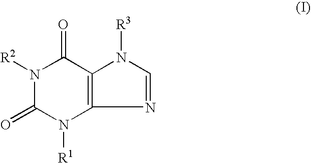

or a salt thereof, (b) a buffer and (c) at least one member selected from sorbic acid, ethylenediaminetetraacetic acid, and salts thereof.

By mixing Ingredients (a), (b) and (c) in combination, the preservative properties exhibited by the sorbic acid or ethylenediaminetetraacetic acid or salts thereof of Ingredient (c) and the preservative properties exhibited by the Compound (I) or salts thereof of Ingredient (a) are further enhanced, thereby enabling a composition to be obtained that advantageously suppresses the generation and proliferation of microorganisms even when the composition is stored for a long period of time.

In the compound represented by Formula (I) in Ingredient (a), R¹, R² and R³ individually represent hydrogen or optionally substituted alkyl. They can be the same group or different.

Examples of alkyl groups represented by R¹, R² and R³ include methyl, ethyl, propyl, isopropyl, butyl, isobutyl, t-butyl and like lower alkyl groups having 1 to 6 carbons, and preferably 1 to 4 carbons. Most preferable alkyl groups are methyl and ethyl groups.

These alkyl groups may have a substituent. Examples of substituents include halogen atoms (for example, chlorine, bromine, and fluorine atoms, etc.); hydroxyl groups; alkoxy groups (for example, methoxy, ethoxy, propoxy, butoxy and like lower alkoxy groups having 1 to 4 carbons); aryloxy groups; carboxyl groups; alkoxycarbonyl groups (for example, alkoxycarbonyl groups having a lower alkoxy group with 1 to 4 carbons) and aryloxycarbonyl groups; acyl groups (for example, formyl, acetyl, propionyl, butyryl, isobutyryl, valeryl and like carbonyl groups having hydrogen or a lower alkyl group with 1 to 4 carbons and carbonyl groups having benzoyl or like aryl substituents); nitro groups; amino groups; and N-substituted amino groups (for example, mono- or di- C₁₋₄ alkylamino group) and cyano groups.

Specific examples of the compounds represented by Formula (I) (Compound (I)) include caffeine, theophylline, oxtriphylline, dyphylline, diisobutylaminobenzoyloxypropyl theophylline, theobromine, diprophylline, proxyphylline, pentoxifylline and like xanthine derivatives. Preferable are caffeine, theophylline, theobromine and like methylxanthine derivatives; diprophylline, proxyphylline, pentoxifylline and the like. Caffeine is particularly preferred. Caffeine includes anhydrous caffeine.

Compound (I) can be used in salt form, and preferably in pharmacologically (pharmaceutically) or physiologically acceptable salt form. Examples of pharmacologically or physiologically acceptable salts herein include organic acid salts (for example, lactates, acetates, butylates, trifluoroacetates, fumarates, maleates, tartrates, citrates, succinates, malonates, methanesulfonates, toluenesulfonates, tosylates, palmitates, stearates and the like); inorganic acid salts (for example, hydrochlorides, sulfates, nitrates, hydrobromides, phosphates and the like); salts with organic bases (for example, salts with methylamine, triethylamine, triethanolamine, morpholine, piperazine, pyllorizine, amino acids, tripyridine, picoline and like organic amines); salts with inorganic bases (for example, ammonium salts; salts with sodium, potassium and like alkali metals; salts with calcium, magnesium and like alkali earth metals; salts with aluminum and like metals; etc.); and the like.

In the composition of the present invention, Compounds (I) and the salts thereof (hereinafter sometimes collectively referred to as “xanthines”) can be used alone or as a combination of two or more species. Further, Compounds (I) can be used in mixture form with other compounds. Examples of such mixtures include caffeine-sodium benzoate, which is a mixture of caffeine and sodium benzoate; caffeine citrate, which is a mixture of caffeine and citric acid; aminophylline, which is a mixture of theophylline and ethylenediamine; ambuphylline, which is a mixture of theophylline and aminoisobutanol; theobromine calcium salicylate and theobromine sodium salicylate, which are mixtures of theobromine and salicylates; theobromine sodium acetate, which is a mixture of theobromine and sodium acetate; and the like.

The proportion of xanthine contained in the composition of the invention varies according to the type of xanthine used and cannot specifically be stated. However, it is usually suitably adjusted to be within the range of 0.0001 to 10% according to the type. A preferable concentration is from 0.001 to 10%, a more preferable concentration is from 0.01 to 5%, a still more preferable concentration is from 0.01 to 3%, and an especially preferable concentration is from 0.1 to 3%.

The buffers of Ingredient (b) are not limited insofar as they exhibit a buffering effect. Specific examples include borate buffers, phosphate buffers, carbonate buffers, citrate buffers, acetate buffers, etc. Preferable are borate buffers, phosphate buffers, carbonate buffers and citrate buffers. More preferable are borate buffers and phosphate buffers. Particularly preferable are phosphate buffers.

The borate buffers herein comprise boric acid; or an alkali metal salt (sodium salt, potassium salt) or an alkali earth metal salt (calcium salt, magnesium salt) of boric acid or the like. The borate buffers can be composed of a mixture of boric acid and a borate. Specific examples of borates include sodium borate, disodium tetraborate and the like.

The phosphate buffers herein comprise phosphoric acid; an alkali metal salt (sodium salt, potassium salt) or alkali earth metal salt (calcium salt, magnesium salt) of phosphoric acid or the like; or a hydrogen phosphate (an alkali metal salt or alkali earth metal salt). The phosphate buffer can be a mixture of two or three species selected from phosphoric acid, phosphates and hydrogen phosphates. Specific examples of hydrogen phosphates include disodium hydrogenphosphate, sodium dihydrogenphosphate, potassium dihydrogenphosphate and the like.

The carbonate buffers herein comprise carbonic acid; an alkali metal salt (sodium salt, potassium salt) or alkali earth metal salt (calcium salt, magnesium salt) of carbonic acid or the like; or a hydrogen carbonate (an alkali metal salt or alkali earth metal salt). The carbonate buffer can be a mixture of two or three species selected from carbonic acid, carbonates and hydrogen carbonates. Specific examples of hydrogen carbonates include sodium hydrogen carbonate, potassium hydrogen carbonate and the like.

The citrate buffers herein comprise citric acid; or an alkali metal salt (sodium salt, potassium salt) or alkali earth metal salt (calcium salt, magnesium salt) of citric acid or the like. The citrate buffer can be a mixture of citric acid and a citrate. Specific examples of citrates include sodium citrate, potassium citrate and the like.

In the buffers described above, borates, phosphates, hydrogen phosphates, carbonates, hydrogen carbonates, citrates and like acid salts can be used in hydrate form.

The above buffers can be used alone or in combination of two or more species.

The proportion of the buffer used can be suitably selected relative to the proportion of the xanthine contained in the composition of the present invention. For example, the buffer is used in a proportion of 0.1 to 500 parts by weight, calculated as the total amount of the acids and the salts thereof described above, preferably in a proportion of 0.1 to 100 parts by weight, and more preferably in a proportion of 0.1 to 50 parts by weight, per part by weight of the xanthine contained in a final formulation.

The concentration of the buffer contained in the composition of the present invention is usually about 0.0001 to about 10%, preferably about 0.001 to about 5%, and more preferably about 0.01 to about 3% calculated as the total content of the acids and the salts thereof described above.

The composition of the present invention comprises as Ingredient (c) at least one member selected from sorbic acid, ethylenediaminetetraacetic acid, and salts thereof.

The sorbic acid can be used either in acid (free form) or salt form, and it can be used as a mixture of the acid and its salt. Preferable examples of sorbates usable herein include alkali metal salts of sorbic acid, which are used as preservatives for ophthalmic compositions, such as potassium sorbate, sodium sorbate and the like.

The ethylenediaminetetraacetic acid (hereinafter sometimes simply referred to as “EDTA”) can be used, similar to the sorbic acid, in acid (free form), salt or hydrate form, and it can also be used as a mixture of the acid and its salt. Preferable examples of the salts of ethylenediaminetetraacetic acid usable herein include alkali metal salts of EDTA such as sodium ethylenediaminetetraacetic acid, disodium ethylenediaminetetraacetic acid, tetrasodium ethylenediaminetetraacetic acid and the like. Disodium ethylenediaminetetraacetic acid dihydrate can be preferably used in the present invention (herein sometimes referred to as “sodium edetate”).

The sorbic acid, EDTA, and salts of these acids can be used alone or in any combination. To prepare a composition having significantly high preservative properties, it is preferable to use the sorbic acid or a salt thereof in combination with the ethylenediaminetetraacetic acid or a salt thereof.

When the sorbic acid or a salt thereof is used, the total proportion thereof is in a range of 0.002 to 100 parts by weight, preferably 0.01 to 10 parts by weight, more preferably 0.01 to 2 parts by weight and still more preferably 0.02 to 2 parts by weight per part by weight of the xanthine contained in the composition. The proportion of the sorbic acid or a salt thereof contained in the composition of the present invention is not limited insofar as it falls within the proportions described above. It can, for instance, be selected according to the application of the composition to comply with legal regulations in various fields concerning pharmaceuticals, quasi-medical products, cosmetics, etc. For example, when the composition is used as a pharmaceutical, especially as a pharmaceutical suitable for application to the skin or mucous membranes, the total concentration of the sorbic acid or a salt thereof in the composition is usually in a range of about 0.00005 to about 10%, preferably about 0.0001 to about 10%, more preferably about 0.0005 to about 5% and still more preferably about 0.001 to about 5%. For quasi-medical products and cosmetics, the concentration of the sorbic acid or its salts described previously can be similarly used.

When the EDTA or a salt thereof is used, the total proportion thereof is 0.001 to 1,000 parts by weight, preferably 0.002 to 100 parts by weight, more preferably 0.002 to 10 parts by weight and still more preferably 0.004 to 1 part by weight per part by weight of the xanthine contained in the composition. The proportion of the EDTA or a salt thereof contained in the composition of the present invention is not limited insofar as it falls within the proportions described above. It can, for instance, be selected according to the application of the composition to comply with usual practice of various fields such as pharmaceuticals, quasi-medical products, cosmetics, etc. For example, the total concentration of the EDTA or a salt thereof in the composition is usually about 0.001 to about 1%, preferably about 0.005 to about 0.5%, more preferably about 0.01 to about 0.3% and still more preferably about 0.01 to about 0.2%.

When the sorbic acid or a salt thereof is used in combination with the EDTA or a salt thereof, the total proportion of EDTA or a salt thereof is, although not limited to, 0.001 to 100 parts by weight, preferably 0.01 to 50 parts by weight and more preferably 0.1 to 5 parts by weight per part by weight of the total amount of the sorbic acid or a salt thereof.

By containing the aforementioned ingredients, the composition of the present invention by itself exhibits exquisite preservative properties. The preservative properties of the composition of the invention can be characterized by the effect of inhibiting the generation and proliferation of any microorganism among Staphylococcus aureus, Pseudomonas aeruginosa, Clostridium sporogenes, Bacillus subtilis, Escherichia coli, Candida albicans, Aspergillus niger, Micrococcus luteus, Bacteroides vulgatus, Serratia marcescens and Fusarium solani. Among these microorganisms, it is preferable to have the effect of inhibiting the generation and proliferation of Staphylococcus aureus, Pseudomonas aeruginosa, Clostridium sporogenes, Bacillus subtilis, Escherichia coli, Candida albicans or Aspergillus niger. Insofar as the composition of the present invention exhibits such preservative properties, the form and application thereof are not limited. The composition can, therefore, be used in a variety of applications for pharmaceuticals, quasi-medical products, cosmetics, (miscellaneous) articles for everyday use, etc., and in a variety of forms.

For safety reasons, it is preferable not to incorporate into the composition of the present invention large amounts of preservatives having generally strong toxicity to tissues and cells, for example benzalkonium chloride, benzethonium chloride and like quaternary ammonium salts, or biguanide compounds such as chlorhexidine. The composition of the present invention does not preclude the use of such preservatives, but it is a composition such that it provides the desired preservative efficacy sufficiently without containing them. Further, with regard to sorbic acid and salts thereof, which pose problems with irritation to the skin and mucous membranes when used alone, the composition of the present invention moderates the irritation by combination with xanthine. Thus, taking advantage of its safe and superior preservative properties, the composition of the present invention can be applied to externally-applied pharmaceuticals and quasi-medical products that are suitable for application to the skin, mucous membranes and the like; internally-applied pharmaceuticals and quasi-medical products that are administered orally, or food compositions; and various other compositions such as articles for daily use, miscellaneous articles and the like.

Insofar as the effects of the invention are not impaired, the composition of the invention can comprise in addition to the ingredients described above a variety of active or medicinal components (including pharmacoactive components and bioactive components) according to the purpose. The type of such components is not limited. For example, when the composition of the present invention is used as an external pharmaceutical or quasi-medical product that is applied to the skin, mucous membranes and the like, examples thereof include congestion-removing components (vasoconstrictors or sympathomimetics), anti-inflammatory components, antihistamic or antiallergic components, astringent components, antibacterial or bactericidal components, vitamins, amino acids, saccharides, topical anesthetic components, steroids, α-adrenergic components, ocular muscle-conditioning components and the like. Although not limited thereto, examples of these components are as follows:

Congestion-removing components (vasoconstrictors or sympathomimetics): epinephrine, ephedrine, tetrahydrozoline, naphazoline, phenylephrine, methylephedrine, and pharmacologically acceptable salts thereof.

Anti-inflammatory components: celecoxib, rofecoxib, indomethacin, diclofenac, pranoprofen, piroxicam, meloxicam, ε-aminocaproic acid, berberine, glycyrrhizinic acid, lysozyme, methyl salicylate, allantoin, and pharmacologically acceptable salts thereof (for example, berberine chloride, berberine sulfate, diclofenac sodium, dipotassium glycyrrhizinate, ammonium glycyrrhizinate, lysozyme chloride, etc.).

Antihistamic or antiallergic components: chlorpheniramine, diphenhydramine, iproheptine, ketotifen, emedastine, clemastine, azelastine, levocabastine, olopatadine, cromoglycic acid, tranilast, amlexanox, mequitazine, loratadine, fexofenadine, cetirizine, ibudilast, suplatast, pemirolast, and pharmacologically acceptable salts thereof (for example, chlorpheniramine maleate, diphenhydramine hydrochloride, iproheptine hydrochloride, ketotifen fumarate, emedastine fumarate, clemastine fumarate, azelastine hydrochloride, levocabastine hydrochloride, olopatadine hydrochloride, sodium cromoglycate, etc.).

Astringent components: zinc and salts thereof (for example, zinc sulfate, zinc lactate).

Antibacterial or bactericidal components: sulfonamides such as sulfamethoxazole, sulfisoxasol, sulfisomidine, and pharmacologically acceptable salts thereof (for example, sodium sulfamethoxazole, sodium sulfisomidine, etc.); acrinol, quaternary ammonium compounds such as benzalkonium, benzethonium, cetylpyridinium, and pharmacologically acceptable salts thereof (for example, benzalkonium chloride, benzethonium chloride, cetylpyridinium chloride, cetylpyridinium bromide, etc.); alkyldiaminoethylglycine hydrochloride; newquinolones such as lomefloxacin, levofloxacin, ciprofloxacin, ofloxacin, norfloxacin, ciprofloxacin chloride, etc.; biguanides such as polyhexamethylene biguanide, chlorhexidine and salts thereof; berberine and salts thereof; polyquatemium-1, Glokill (trade name, product of Rhodia Japan, Ltd.), polydiallyldimethyl ammonium chloride, poly[oxyethylene(dimethyliminio)ethylene-(dimethyliminio)ethylenedichloride]; and parabens (methyl paraben, ethyl paraben, and salts thereof).

Vitamins: vitamins A such as retinal, retinol, retinoic acid, carotene, dehydroretinal, lycopene, and pharmacologically acceptable salts thereof (for example, retinol acetate, retinol palmitate, etc.) and the like; vitamins B such as thiamine, thiamine disulfide, dicethiamine, octothiamine, cycothiamine, bisibuthiamine, bisbenthiamine, prosulthiamine, benfothiamine, fursulthiamine, riboflavin, flavin adenine dinucleotide, pyridoxine, pyridoxal, hydroxocobalamin, cyanocobalamin, methylcobalamin, deoxyadenocobalamin, folic acid, tetrahydrofolic acid, dihydrofolic acid, nicotinic acid, nicotinic-acid amide, nicotinic alcohol, pantothenic acid, panthenol, biotin, choline, inositol, and pharmacologically acceptable salts thereof (for example, thiamine hydrochloride, thiamine nitrate, dicethiamine hydrochloride, fursulthiamine hydrochloride, riboflavin butyrate, flavin adenine dinucleotide sodium, pyridoxine hydrochloride, pyridoxal phosphate, calcium pyridoxal phosphate, hydroxocobalamin hydrochloride, hydroxocobalamin acetate, calcium pantothenate, sodium pantothenate, etc.) and the like; vitamins C such as ascorbic acid and derivatives thereof, erythorbic acid and derivatives thereof, and pharmacologically acceptable salts thereof (for example, sodium ascorbate, sodium erythorbate, etc.) and the like; vitamins D such as ergocalciferol, cholecalciferol, hydroxycholecalciferol, dihydroxycholecalciferol, dihydrotachysterol, and pharmacologically acceptable salts thereof, etc.; vitamins E such as tocopherol and derivatives thereof, ubiquinone derivatives, and pharmacologically acceptable salts thereof (tocopherol acetate, tocopherol nicotinate, tocopherol succinate, tocopherol calcium succinate, etc.) and the like; and other vitamins such as carnitine, ferulic acid, γ-oryzanol, orotic acid, rutin, eriocitrine, hesperidin, and pharmacologically acceptable salts thereof (carnitine chloride, etc.).

Amino acids: leucine, isoleucine, valine, methionine, threonine, alanine, phenylalanine, tryptophan, lysine, glycine, asparagine, aspartic acid, serine, glutamine, glutamic acid, proline, tyrosine, cystein, histidine, ornithine, hydroxyproline, hydroxylysine, glycylglycine, aminoethylsulfonic acid (taurine), and their pharmacologically acceptable salts (for example, potassium aspartate, magnesium aspartate, cysteine hydrochloride, etc.).

Saccharides: monosaccharides (glucose and the like), disaccharides (trehalose, lactose, fructose and the like), oligosaccharides (lactulose, raffinose, pullulan and the like), cellulose and derivatives thereof (methylcellulose, ethylcellulose, hydroxyethylcellulose, hydroxypropylcellulose, carboxymethylcellulose, carboxyethylcellulose, nitrocellulose and the like), high-molecular-weight saccharides (chondroitin sulfate, hyaluronic acid and the like) and pharmacologically acceptable salts thereof (sodium chondroitin sulfate, sodium hyaluronate, etc.), and sugar alcohols (mannitol, xylitol, sorbitol, etc.).

Topical anesthetic components: lidocaine, oxyprocaine, dibucaine, procaine, ethyl aminobenzoate, meprylcaine and salts thereof (lidocaine hydrochloride, oxybuprocaine hydrochloride, etc.).

Steroids: hydrocortisone, prednisolone, and salts thereof.

α-Adrenergic components: imidazoline derivatives (naphazoline, tetrahydrozoline, etc.), β-phenylethylamine derivatives (phenylephrine, epinephrine, ephedrine, methylephedrine, etc.), and pharmaceutically or physiologically acceptable salts thereof (for example, naphazoline hydrochloride, naphazoline nitrate, tetrahydrozoline hydrochloride, tetrahydrozoline nitrate, phenylephrine hydrochloride, epinephrine hydrochloride, ephedrine hydrochloride, methylephedrine hydrochloride and similar inorganic acid salts, epinephrine bitartrate and similar organic acid salts).

Ocular muscle-conditioning components: choline esterase inhibitors that have an active center similar to that of acetylcholine, for example, neostigmine methylsulfate and like quaternary ammonium compounds, and salts thereof.

Other components: polyvinyl alcohols (including completely or partially saponified compounds), polyvinyl pyrrolidone, etc.

The proportion of these components that can be incorporated into an externally-applied composition can be selected according to the purpose of the composition, the type of active components to be used, etc. Although not limited, the total concentration of these components is suitably selected from a range of about 0.0001 to about 30% and preferably about 0.001 to 10% relative to the entire externally-applied composition. More specifically, suitable examples of the concentrations of respective components that can be incorporated into the externally-applied aqueous composition of the present invention are as follows:

Congestion-removing components (vasoconstrictors or sympathomimetics): 0.0001 to 0.5%, preferably 0.0005 to 0.3% and more preferably 0.001 to 0.1%.

Anti-inflammatory components: 0.0001 to 10% and preferably 0.0001 to,5%.

Antihistamic or antiallergic components: 0.0001 to 10% and preferably 0.001 to 5%.

Astringent components: 0.0001 to 10% and preferably 0.005 to 2.5%.

Antibacterial or bactericidal components: 0.001 to 10% and preferably 0.01 to 10%.

Vitamins: 0.0001 to 1% and preferably 0.0001 to 0.5%

Amino acids: 0.0001 to 10% and preferably 0.001 to 3%.

Saccharides (monosaccharides, disaccharides, oligosaccharides and sugar alcohols): 0.0001 to 5%, preferably 0.001 to 5% and more preferably 0.01 to 2%.

High-molecular-weight saccharides and salts thereof: 0.0001 to 2%, preferably 0.01 to 2% and more preferably 0.01 to 1%.

Cellulose and derivatives thereof: 0.001 to 5%. and preferably 0.01 to 1%.

Topical anesthetic components: 0.0001 to 2% and preferably 0.001 to 1%.

Steroids: 0.0001 to 10% and preferably 0.001 to 3%.

α-Adrenergic components: 0.0001 to 2% and preferably 0.001 to 1%.

Ocular muscle-conditioning components: 0.0001 to 0.5% and preferably 0.001 to 0.1%.

Polyvinyl pyrrolidone and polyvinyl alcohols: 0.001 to 10%, preferably 0.001 to 5% and more preferably 0.01 to 3%.

Insofar as the effects of the invention are not impaired, various other components and additives can be further incorporated as required into the composition of the invention using known methods according to the purpose and form of the composition. Examples of additives include antioxidants, colorants, flavorings, cooling agents, solubilizers, suspending agents, emulsifiers, isotonicity adjusters, buffers, thickeners, pH adjusters, chelating agents, inorganic salts, etc. Among these, solubilizers are preferred for improving the dissolution stability of the xanthine incorporated into the composition of the invention, thereby making the composition stable. Examples of such solubilizers include benzoic acid, citric acid, aminoisobutanol, taurine and salts thereof, various surfactants, propylene glycol and like polyalcohols, and the like.

The composition of the present invention is not limited by the form or properties thereof. The composition may be in the form of a solid, semisolid or liquid, and may be aqueous or oil based. Preferably, the composition is an aqueous composition in the form of a semisolid or liquid. The composition can be prepared using a suitable base according to the form of the composition in solid, semisolid or liquid. For example, when a semisolid formulation, especially an externally-applied formulation (a composition that is suitable for application to the skin or mucous membranes), is prepared, an ointment base (for example, petrolatum, liquid paraffin, wax or another hydrocarbon base, cetanol, ester of higher fatty acid, or the like), gel base (for example, carboxyvinyl polymer, polyoxyethylene polyoxypropylene block copolymer, gum, or the like), or oil base (olive oil, soybean oil, sesame oil, cotton seed oil or like vegetable oil, propyleneglycol, or the like) can be used. When a liquid formulation is prepared, an aqueous base, such as water, aqueous solvent or the like, or an oil base, such as an oil solvent or the like, can be used.

Examples of the composition of the present invention include aqueous compositions in various forms (for example, liquid, lotion, cream, ointment, gel, suspension, emulsion, extract, air sol, etc.) that are suitable for application to the skin or mucous membranes in a variety of fields such as pharmaceuticals, quasi-medical products, cosmetics and articles for daily use (miscellaneous articles).

Although sorbic acid and salts thereof have heretofore been used as preservatives that are relatively safe compared with benzalkonium chloride and like cationic surfactant preservatives, they pose the problem of irritating the skin, especially to the mucous membranes. Although the compositions that fall within the scope of the present invention include compositions containing sorbic acid or a salt thereof that are applied to the skin and mucous membranes, these compositions moderate the irritation of sorbic acid and salts thereof by the addition of xanthine. Therefore, the composition (hereinafter sometimes referred to as “compositions for the skin” or “compositions for the mucous membranes”) of the present invention can be suitably applied to the skin and especially to the lips and mucous membranes (such as cornea, conjunctiva and like ocular, oral, nasal, pharyngeal mucous membranes, etc.), which are irritation sensitive. The compositions for the mucous membranes herein refer to those directly applied to the mucous membranes, for example, ophthalmic compositions such as ophthalmic solutions (including those usable while contact lenses (CL) are in use), collyriums (including contact lens (CL)-friendly collyriums usable while contact lenses are in use), ophthalmic ointment and contact lens-wearing solutions; ear and nasal compositions (such as nasal drops, ear drops, nasal washing fluid); and oral compositions (such as oropharyngeal formulations, mouthwash, etc.) as well as those in the form in which they are not directly applied to the mucous membranes but could be applied to or contact the mucous membranes under certain circumstances, for example, contact lens-care formulations (such as cleaning solutions, soaking solutions, disinfecting solutions, multi-purpose solutions, etc.), cosmetics (such as eyeliner, lipstick, lip cream, etc.), and the like.

Typical ingredients that are suitable for incorporation into the compositions for the mucous membranes, especially aqueous compositions for the mucous membranes, are set forth below, although the scope of the present invention is not limited by these ingredients:

Thickeners: polysaccharides and derivatives thereof (such as gum arabic, karaya gum, xanthan gum, carob gum, guar gum, guaiac, quince seed, Dammar resin, tragacanth, benzoin gum, locust bean gum, casein, agar, alginic acid, dextrin, dextran, carrageenan, gelatin, collagen, pectin, starch, polygalacturonic acid, chitin and its derivatives, chitosan and its derivatives, elastin, heparin, heparinoid, heparin sulfate, heparan sulfate, hyaluronic acid, chondroitin sulfate, etc.), ceramide, cellulose derivatives (methylcellulose, ethylcellulose, hydroxyethyl cellulose, hydroxypropyl cellulose, hydroxypropylmethyl cellulose, carboxymethyl cellulose, carboxyethyl cellulose, cellulose, nitro cellulose, etc.), polyvinyl alcohols (including completely or partially saponified compounds), polyvinyl pyrrolidone, macrogol, polyvinyl meta-acrylate, polyacrylic acid, carboxyvinyl polymer, polyethyleneimine, ribonucleic acid, deoxyribonucleic acid and the pharmacologically acceptable salts thereof.

Surfactants: non-ionic surfactants such as polyoxyethylene (POE)-polyoxypropylene (POP) block copolymers, (for example, poloxamer 407, poloxamer 235, poloxamer 188, etc.), POE (20) sorbitan monolaurates (polysorbate 20), POE (20) sorbitan monooleates (polysorbate 80) and like POE sorbitan fatty acid esters, POE (60) hydrogenated castor oil and like POE hydrogenated castor oils, POE (9) lauryl ether and like POE alkyl ethers, POE (20) POP (4) cetyl ether and like POE-POP alkyl ethers, POE (10) nonylphenyl ether and like POE alkylphenyl ethers and the like; ampholytic surfactants such as alkyl diamino ethyl glycine and like glycine surfactants, lauryl dimethyl amino betaine acetate and like betaine acetate surfactants, imidazoline surfactants and the like; anionic surfactants such as sodium POE (10) lauryl ether phosphate, and like POE alkyl ether phosphates and their salts, sodium lauroyl methyl alanine and like salts of N-acylamino acid, alkylether carboxylates, sodium N-cocoylmethyltaurate and like N-acyltaurates, sodium tetradecene sulfonate and like sulfonates, sodium lauryl sulfate and like alkyl sulfates, sodium POE (3) lauryl ether sulfate and like POE alkyl ether sulfates, α-olefin sulfonate and the like; cationic ion surfactants such as alkyl amine salts, alkyl quaternary ammonium salts (benzalkonium chloride, benzethonium chloride, etc.), alkylpyridinium salts (cetylpyridinium chloride, cetylpyridinium bromide, etc.) and the like. (The numbers in the parentheses herein represent the molar number of ethylene oxide added.)

Preservatives, bactericidal agents and antibacterial agents: p-hydroxybenzoate esters (methyl parahydroxybenzoate, ethyl parahydroxybenzoate, propyl parahydroxybenzoate, butyl parahydroxybenzoate, etc.), acrinol, methyl rosaniline chloride, benzalkonium chloride, benzethonium chloride, cetylpyridinium chloride, cetylpyridinium bromide, chlorhexidine, polyhexamethylene biguanide, alkyldiyaminoethylglycine hydrochloride, benzylalcohol, phenethyl alcohol, chlorobutanol, isopropanol, ethanol, phenoxyethanol, sulfur, zirconium phosphate with silver, zinc, or zinc oxide, silver zinc alminosilicate, mercurochrome, thimerosal, povidone-iodine, dehydroacetic acid, chlorxylenol, cresol, chlorophen, phenol, resorcin, orthophenyl phenol, isopropyl methylphenol, thymol, hinokitiol, sulfamine, lysozyme, lactoferrin, triclosan, 8-hydroxy quinoline, undecylenic acid, capric acid, propionic acid, benzoic acid, propionic acid, sorbic acid, triclocarban sorbate, halocarban, thiabendazole, polymyxin B, 5-chloro-2-methyl-4-isothiazoline-3-one,2-methyl-4-isothiazoline-3-one, polylysine, hydrogen peroxide, polyquatemium-1, Glokill (trade name, product of Rhodia Japan, Ltd., for example Glokill PQ etc.), polydiallyldimethyl ammonium chloride, poly[oxyethylene(dimethyliminio)ethylene-(dimethyliminio)ethylenedichloride], and their pharmacologially acceptable salts.

pH-adjusters: inorganic acids (hydrochloric acid, sulfuric acid, phosphoric acid, polyphosphoric acid, boric acid, etc.), organic acids (lactic acid, acetic acid, citric acid, tartaric acid, malic acid, succinic acid, oxalic acid, gluconic acid, fumaric acid, propionic acid, acetic acid, aspartic acid, ε-aminocaproic acid, glutamic acid, aminoethylsulfonic acid, etc.), gluconolactone, ammonium acetate, inorganic bases (sodium hydrogen carbonate, sodium carbonate, potassium hydroxide, sodium hydroxide, calcium hydroxide, magnesium hydroxide, etc.), organic bases (monoethanolamine, triethanolamine, diisopropanolamine, triisopropanolamine, lysine, etc.), borax, and their pharmacologically acceptable salts and the like.

Isotonicity adjusters: glycerin, propyleneglycol and like polyalcohols, saccharides (glucose, mannitol, sorbitol, etc.), and the like.

Chelating agent: edetic acids (such as ethylenediaminetetraacetic acid, i.e., EDTA), ethylenediaminediacetic acid (EDDA), diethylenetriaminepentaacetic acid (DTPA), N-(2-hydroxyethyl)ethylenediaminetriacetic acid (HEDTA), N-(2-hydroxyethyl)iminodiacetic acid (HIDA), citric acid, tartaric acid, phosphoric acids (polyphosphoric acid, hexametaphosphoric acid, metaphosphotic acid), succinic acid, trihydroxymethylaminomethane, nitrilotriacetic acid, 1-hydroxyethane-1,1-diphosphonic acid, etc.

Inorganic salts: sodium chloride, potassium chloride, sodium carbonate, sodium hydrogen carbonate, calcium chloride, magnesium sulfate, sodium dihydrogenphosphate, disodium hydrogenphosphate, dipotassium hydrogenphosphate, sodium thiosulphate, sodium acetate, etc.

Flavorings and cooling agents: menthol, camphor, borneol, geraniol, eucalyptus oil, bergamot oil, fennel oil, mentha oil, cinnamon oil, rose oil, peppermint oil, etc.

The composition of the present invention can be prepared to have a biologically acceptable pH and/or osmotic pressure as necessary or according to the form, purpose or administration method.

When prepared as a composition for the skin, the composition of the invention has a pH in a range of 2 to 10, and for low irritation and good sensation to the skin, the pH is preferably in the range of 3 to 9 and more preferably in the weakly acidic to neutral range of 5 to 8. When the composition is prepared as a composition for the mucous membranes such as an ophthalmic formulation, collyrium or the like, the pH is usually in the range of 4 to 9; preferable is 5 to 8.5 and particularly preferable is 5.5 to 8.5.

When prepared as a composition for the mucous membranes, the composition of the invention has an osmotic pressure of about 100 to about 1,200 mOsm, preferably about 100 to about 600 mOsm and particularly preferably about 150 to about 400 mOsm; and the ratio of the osmotic pressure to the physiological saline solution is in a range of about 0.3 to 4.1, preferablely about 0.3 to about 2.1 and particularly preferably about 0.5 to about 1.4. Osmotic pressure and pH can be adjusted by using buffers, pH adjusters, isotonicity adjusters, salts, etc.

The composition of the present invention can be prepared in the desired form according to known methods employing the aforementioned essential ingredients and other optional components as necessary. For example, semisolid formulations and liquid formulations can be prepared by mixing various bases with respective ingredients, adjusting the osmotic pressure and pH of the mixture as necessary and inserting it into a vessel.

(2) Method for Enhancing Preservative Properties.

(2-1) The present invention provides a method for synergistically enhancing the preservative properties of (c) sorbic acid, ethylenediaminetetraacetic acid (EDTA), or a salt thereof, or the preservative properties of (a) a xanthine, which are known as preservatives, by combining (a) a xanthine, (b) a buffer and (c) at least one member selected from sorbic acid, EDTA, and salts thereof.

The xanthines of Ingredient (a), buffers of Ingredient (b) and sorbic acid, EDTA, and their salts of Ingredient (c) usable herein are those specifically described in (1) above. The method of the present invention wherein Ingredients (a), (b) and (c) are used in combination is not limited insofar as these ingredients can coexist.

The proportions for using these ingredients are also as disclosed in (1) above. Specifically, when the sorbic acid or a salt thereof is used as Ingredient (c), the xanthine can be used in a proportion of 0.01 to 500 parts by weight per part by weight of the total amount of the sorbic acid or its salt, preferably 0.1 to 100 parts by weight, more preferably 0.5 to 100 parts by weight and still more preferably 0.5 to 50 parts by weight; and per part by weight of xanthine the buffer can be used in a proportion of 0.1 to 500 parts by weight, preferably in a proportion of 0.1 to 100 parts by weight and more preferably in a proportion of 0.1 to 50 parts by weight. When the EDTA or a salt thereof is used as Ingredient (c), the xanthine can be used in a proportion of 0.001 to 1,000 parts by weight per part by weight of the total amount of the EDTA or its salt, preferably in a proportion of 0.01 to 500 parts by weight, more preferably in proportion of 0.1 to 500 parts by weight and still more preferably in a proportion of 1 to 250 parts by weight; and per part by weight of xanthine the buffer can be used in a proportion of 0.1 to 500 parts by weight, preferably in a proportion of 0.1 to 100 parts by weight and more preferably in a proportion of 0.1 to 50.

The method of the present invention for enhancing preservative properties is to enhance the preservative properties of sorbic acid, EDTA, and their salts or the preservative properties of xanthine. Therefore, insofar as these objectives are achieved, the present invention does not limit the types of microorganisms whose proliferation is to be controlled. Examples of such microorganisms are those microorganisms selected from Staphylococcus aureus, Pseudomonas aeruginosa, Clostridium sporogenes, Bacillus subtilis, Escherichia coli, Candida albicans, Aspergillus niger, Micrococcus luteus, Bacteroides vulgatus, Serratia marcescens and Fusarium solani to which sorbic acid, EDTA, and their-salts exhibit the effect of inhibiting their proliferation or to which xanthines exhibit the effect of inhibiting their proliferation. Preferable are those microorganisms selected from Staphylococcus aureus, Pseudomonas aeruginosa, Clostridium sporogenes, Bacillus subtilis, Escherichia coli, Candida albicans and Aspergillus niger to which sorbic acid, EDTA, and their salts exhibit the effect of inhibiting their proliferation or to which xanthines exhibit the effect of inhibiting their proliferation.

(2-2) The present invention provides a method for further enhancing the preservative properties of (c) sorbic acid, ethylenediaminetetraacetic acid (EDTA), or a salt thereof, which is known as preservatives, by using (c) at least one member selected from sorbic acid, EDTA, and their salts, in combination with (a) a xanthine and (b) a buffer.

The xanthines of Ingredient (a), buffers of Ingredient (b) and sorbic acid, EDTA, and their salts of Ingredient (c) usable herein are those specifically described in (1) above. The method of the present invention wherein Ingredient (c) is used in combination with Ingredients (a) and (b) is not limited insofar as these ingredients can coexist.

The proportions for using these ingredients are as disclosed in (1) above. Specifically, when the sorbic acid or a salt thereof is used as Ingredient (c), the xanthine can be used in a proportion of 0.01 to 500 parts by weight per part by weight of the total amount of the sorbic acid and a salt thereof, preferably in a proportion of 0.1 to 100 parts by weight, still preferably in a proportion of 0.5 to 100 parts by weight and more preferably in a proportion of 0.5 to 50 parts by weight; and per part by weight of xanthine the buffer of Ingredient (b) can be used in a proportion of 0.1 to 500 parts by weight, preferably in a proportion of 0.1 to 100 parts by weight and more preferably in a proportion of 0.1 to 50 parts by weight. When the EDTA or a salt thereof is used as Ingredient (c), the xanthine can be used in a proportion of 0.001 to 1,000 parts by weight per part by weight of the total amount of the EDTA and a salt thereof, preferably in a proportion of 0.01 to 500 parts by weight, more preferably in a proportion of 0.1 to 500 parts by weight and still more preferably in a proportion of 1 to 250; and per part by weight of xanthine the buffer of Ingredient (b) can be used in a proportion of 0.1 to 500 parts by weight, preferably in a proportion of 0.1 to 100 parts by weight and more preferably in a proportion of 0.1 to 50.

As other embodiments of the method for enhancing the preservative properties of sorbic acid, EDTA, or their salts, the present invention provides the methods described in following Items (i) and (ii).

(i) A method for enhancing the preservative properties of a composition comprising at least one member selected from sorbic acid, EDTA, and their salts:

This method is accomplished through the use of a composition comprising at least one member selected from sorbic acid, EDTA, and their salts, in combination with the aforementioned xanthine and buffer.

When the composition described above contains the sorbic acid or a salt thereof, the total concentration of the sorbic acid and a salt thereof contained in the final formulation obtained by further mixing the xanthine and the buffer with the composition is in a range of 0.00005 to 10%, preferably in a range of 0.0001 to 10%, more preferably in a range of 0.0005 to 5% and still more preferably in a range of 0.001 to 5%. When the composition described above contains the EDTA or a salt thereof, the total concentration of the EDTA and a salt thereof contained in the final formulation obtained by further mixing the xanthine and the buffer with the composition is in a range of 0.001 to 1%, preferably in a range of 0.005 to 0.5%, preferably in a range of 0.01 to 0.3% and still more preferably in a range of 0.01 to 0.2%.

When the composition described above contains both the sorbic acid or a salt thereof, and the EDTA or a salt thereof, the proportion of these ingredients contained in the composition is such that the total amount of the EDTA or a salt thereof is, although not limited to, in a proportion of 0.001 to 100 parts by weight, preferably 0.01 to 50 parts by weight and more preferably 0.1 to 5 parts by weight per part by weight of the total amount of the sorbic acid or a salt thereof.

The proportion of xanthine to be added in the composition described above is, when the composition contains the sorbic acid or a salt thereof, suitably selected from the range of 0.01 to 500 parts by weight, preferably 0.1 to 100 parts by weight, more preferably 0.5 to 100 parts by weight and still more preferably 0.5 to 50 parts by weight per part by weight of the total amount of the sorbic acid or a salt thereof; and when the composition contains the EDTA or a salt thereof, the proportion of xanthine to be added in the composition is suitably selected from the range of 0.001 to 1,000 parts by weight, preferably 0.01 to 500 parts by weight, more preferably 0.1 to 500 parts by weight and still more preferably 1 to 250 parts by weight per part by weight of the total amount of the EDTA or a salt thereof. The proportion of the buffer to be added in the composition can be selected relative to the proportion of xanthine added in the composition. For example, the buffer is suitably used in a proportion of 0.1 to 500 parts by weight, preferably in a proportion of 0.1 to 100 parts by weight and more preferably in a proportion of 0.1 to 50 parts by weight per part by weight of the xanthine contained in the final formulation.

From another perspective, the method described above can be stated as a method for producing a composition having enhanced preservative properties, over preservative properties of compositions comprising at least one member selected from sorbic acid, EDTA, and their salts. Specifically, this production method can be practiced by using a composition comprising as its ingredient at least one member selected from sorbic acid, EDTA, and their salts, in combination with a xanthine and a buffer.

(ii) A method for imparting strong perservative properties to a composition comprising a buffer and at least one member selected from sorbic acid, EDTA, and their salts; or a method for enhancing the preservative properties of such a composition:

This method is accomplished through the use of a composition comprising a buffer and at least one member selected from sorbic acid, EDTA, and their salts, in combination with xanthine.

The proportion of the buffer contained in the composition described above is in a range of 0.0001 to 10%, preferably in a range of 0.001 to 5% and more preferably in a range of 0.01 to 3% based on the final formulation obtained by further mixing xanthine with the composition. When the composition described above comprises the sorbic acid or a salt thereof, the total concentration of the sorbic acid or a salt thereof is in a range of 0.00005 to 10%, preferably in a range of 0.0001 to 10%, more preferably in a range of 0.0005 to 5% and still more preferably in a range of 0.001 to 5% based on the final formulation obtained by further mixing xanthine with the composition. When the composition described above comprises the EDTA or a salt thereof, the total concentration of the EDTA or a salt thereof is in a range of 0.001 to 1%, preferably in a range of 0.005 to 0.5%, more preferably in a range of 0.01 to 0.3% and still more preferably in a range of 0.01 to 0.2% based on the final formulation obtained by further mixing xanthine with the composition.

When the composition described above comprises both the sorbic acid or a salt thereof, and the EDTA or a salt thereof, the proportion of these ingredients contained in the composition is such that the total amount of the EDTA or a salt thereof is, although not limited to, in a proportion of 0.001 to 100 parts by weight, preferably in a proportion of 0.01 to 50 parts by weight and more preferably in a proportion of 0.1 to 5 parts by weight per part by weight of the total amount of the sorbic acid or a salt thereof.

The proportion of xanthine to be added in the composition described above is, when the composition comprises the sorbic acid or a salt thereof, in a range of 0.01 to 500 parts by weight per part by weight of the total amount of the sorbic acid or its salt, preferably in a range of 0.1 to 100 parts by weight, more preferably in a range of 0.5 to 100 parts by weight and still more preferably in a range of 0.5 to 50 parts by weight; and when the composition comprises the EDTA or a salt thereof, the proportion of xanthine to be added in the composition is in a range of 0.001 to 1,000 parts by weight part per by weight of the total amount of the EDTA or its salt, preferably in a range of 0.01 to 500 parts by weight, more preferably in a range of 0.1 to 500 parts by weight and still more preferably in a range of 1 to 250 parts by weight. The proportion of xanthine to be added in the composition is in a range of 0.2 to 1,000 parts by weight, preferably in a range of 1 to 1,000 parts by weight and more preferably in a range of 2 to 1,000 parts relative to 100 parts by weight of the buffer contained in the composition.

From another perspective, the method described above can be stated as a method for producing a composition having enhanced preservative properties, over preservative properties of a composition comprising a buffer and at least one member selected from sorbic acid, EDTA, and their salts as its ingredients. Specifically, this production method can be practiced by using a composition containing as its ingredients a buffer and at least one member selected from sorbic acid, EDTA, and their salts, in combination with xanthine.

The method of the present invention for enhancing preservative properties is to enhance the preservative properties of sorbic acid, EDTA, and their salts, or the preservative properties of compositions comprising these compounds. Therefore, insofar as these objectives are achieved, the present invention does not limit the types of microorganisms whose the proliferation is to be controlled. Examples of such microorganisms are those microorganisms selected from Staphylococcus aureus, Pseudomonas aeruginosa, Clostridium sporogenes, Bacillus subtilis, Escherichia coli, Candida albicans, Aspergillus niger, Micrococcus luteus, Bacteroides vulgatus, Serratia marcescens and Fusarium solani to which sorbic acid, EDTA, or their salts exhibit the effect of inhibiting their proliferation. Preferable are those microorganisms selected from Staphylococcus aureus, Pseudomonas aeruginosa, Clostridium sporogenes, Bacillus subtilis, Escherichia coli, Candida albicans and Aspergillus niger to which sorbic acid, EDTA, or their salts exhibit the effect of inhibiting their proliferation.

(2-3) Moreover, the present invention provides a method for further enhancing the preservative properties of (a) a xanthine by combining (a) a xanthine with (b) a buffer and (c) at least one member selected from sorbic acid, EDTA, and their salts.

The buffers of Ingredient (b), and sorbic acid, EDTA, and their salts of Ingredient (c) usable herein are those specifically described in (1) above. Among the xanthines of Ingredient (a) specifically described in (1), preferable examples are those that intrinsically exhibit preservative properties. The method of the present invention wherein Ingredients (b) and (c) are used in combination with Ingredient (a) is not limited insofar as these ingredients can coexist.

The proportions for using these ingredients are as disclosed in (1) above. Specifically, when the sorbic acid or its salt is used as Ingredient (c), the sorbic acid and a salt thereof can be used in a total proportion of 0.002 to 100 parts by weight, preferably in a proportion of 0.01 to 10 parts by weight, more preferably in a proportion of 0.01 to 2 parts by weight and still more preferably in a proportion of 0.02 to 2 parts by weight per part by weight of xanthine; and the buffer can be used in a proportion of 0.1 to 500 parts by weight, preferably in a proportion of 0.1 to 100 parts by weight and more preferably in a proportion of 0.1 to 50 parts by weight per part by weight of xanthine. When the EDTA or a salt thereof is used as Ingredient (c), the EDTA and its salt can be used in a total proportion of 0.001 to 1,000 parts by weight, preferably in a proportion of 0.002 to 100 parts by weight, more preferably in a proportion of 0.002 to 10 parts by weight and still more preferably in a proportion of 0.004 to 1 part by weight per part by weight of xanthine; and the buffer can be used in a proportion of 0.1 to 500 parts by weight, preferably in a proportion of 0.1 to 100 parts by weight and more preferably in a proportion of 0.1 to 50 parts by weight per part by weight of xanthine.

As other embodiments of the method for enhancing the preservative properties of xanthine, the present invention provides the methods described in following Items (iii) and (iv).

(iii) A method for enhancing the preservative properties of a composition comprising a xanthine:

This method is accomplished through the use of a composition comprising a xanthine in combination with a buffer and at least one member selected from sorbic acid, EDTA, and their salts.

The total concentration of xanthine contained in the composition is usually in a range of 0.0001 to 10%, preferably in a range of 0.001 to 10%, more preferably in a range of 0.01 to 5%, still more preferably in a range of 0.01 to 3%, and most preferably in a range of 0.1 to 3% based on the final formulation obtained by further mixing the sorbic acid, EDTA, or a salt thereof, and the buffer with the composition.

The proportion of the sorbic acid or a salt thereof to be added in the aforementioned composition is in a range of 0.002 to 100 parts by weight, preferably in a range of 0.01 to 10 parts by weight, more preferably in a range of 0.01 to 2 parts by weight and still more preferably in a range of 0.02 to 2 parts by weight per part by weight of the xanthine contained in the composition. The proportion of the EDTA and its salt to be added in the composition is usually in a range of 0.001 to 1,000 parts by weight, preferably in a range of 0.002 to 100 parts by weight, still preferably in a range 0.002 to 10 parts by weight and more preferably in a range of 0.004 to 1 parts by weight per part by weight of the xanthine contained in the composition. Moreover, the proportion of the buffer to be added in the composition is usually in a range of 0.1 to 500 parts by weight, preferably in a range of 0.1 to 100 parts by weight and more preferably in a range of 0.1 to 50 parts by weight per part by weight of the xanthine contained in the composition.

When both the sorbic acid or its salt, and the EDTA or its salt are added in the composition, the EDTA or its salt can be used in a total proportion of 0.001 to 100 parts by weight, preferably 0.01 to 50 parts by weight and more preferably 0.1 to 5 parts by weight per part by weight of the total amount of the sorbic acid or its salt contained in the final formulation.

From another perspective, the method described above can be stated as a method for producing a composition having enhanced preservative properties, over preservative properties of compositions containing a xanthine as their ingredient. Specifically, this production method can be practiced through the use of a composition comprising as its ingredient a xanthine in combination with a buffer and at least one member selected from sorbic acid, EDTA, and their salts.

(iv) A method for imparting strong preservative properties to a composition comprising a xanthine and a buffer; or a method for enhancing the preservative properties of such a composition:

This method is accomplished through the use of a composition comprising a xanthine and a buffer in combination with at least one member selected from sorbic acid, EDTA, and their salts, as described above.

The concentration of xanthine contained in the composition described above is usually in a range of 0.0001 to 10%, preferably in a range of 0.001 to 10%, more preferably in a range of 0.01 to 5%, still more preferably in a range of 0.01 to 3% and most preferably in a range of 0.1 to 3% based on the final formulation obtained by further mixing the sorbic acid, EDTA, or a salt thereof with the composition. The concentration of the buffer contained in the composition is usually in a range of about 0.0001 to about 10%, preferably in a range of about 0.001 to about 5% and more preferably in a range of 0.01 to 3% based on the final formulation obtained by further mixing the sorbic acid, EDTA, or a salt thereof with the composition.

The total proportion of the sorbic acid or a salt thereof to be added in the composition is usually in a range of 0.002 to 10 parts by weight, preferably in a range of 0.01 to 10 parts by weight, more preferably in a range of 0.01 to 2 parts by weight and still more preferably in a range of 0.02 to 2 parts by weight per part by weight of the xanthine contained therein. Moreover, the total proportion of the EDTA or a salt thereof to be added in the composition is usually in a range of 0.001 to 1,000 parts by weight, preferably in a range of 0.002 to 100 parts by weight, more preferably in a range of 0.002 to 10 parts by weight and still more preferably in a range of 0.004 to 1 part by weight per part by weight of the xanthine contained therein.

When both the sorbic acid or its salt, and the EDTA or its salt are added in the composition, the EDTA or its salt can be used in a total proportion of 0.001 to 100 parts by weight, preferably 0.01 to 50 parts by weight and more preferably 0.1 to 5 parts by weight per part by weight of the total amount of the sorbic acid or its salt contained in the final formulation.

From another perspective, the method described above can be stated as a method for producing a composition having enhanced preservative properties, over preservative properties of a composition comprising a xanthine and a buffer. Specifically, this production method can be practiced through the use of a composition comprising as its ingredients a xanthine and a buffer in combination with at least one member selected from sorbic acid, EDTA, and their salts.

The method of the present invention for enhancing preservative properties is to enhance the preservative properties of a xanthine or the preservative properties of a conposition containing a xanthine. Therefore, insofar as these objectives are achieved, the present invention does not limit the types of microorganisms whose proliferation is to be controlled. Examples of such microorganisms are those microorganisms selected from Staphylococcus aureus, Pseudomonas aeruginosa, Clostridium sporogenes, Bacillus subtilis, Escherichia coli, Candida albicans, Aspergillus niger, Micrococcus luteus, Bacteroides vulgatus, Serratia marcescens and Fusarium solani to which xanthines exhibit the effect of inhibiting their proliferation. Preferable examples are those microorganisms selected from Staphylococcus aureus, Pseudomonas aeruginosa, Clostridium sporogenes, Bacillus subtilis, Escherichia coli, Candida albicans and Aspergillus niger to which xanthines exhibit the effect of inhibiting their proliferation.

### EXAMPLES

The present invention is described in more detail with reference to the following examples and test examples, although the scope of the present invention is not limited by these examples.

### Test Example 1

Preservative Efficacy Test (Antiseptic Test)

Test solutions having the ingredients set forth in Tables 1 and 2 were prepared (Examples 1 to 6 and Comparative Examples 1 to 10). A preservative efficacy test (antiseptic test) was conducted for these test solutions according to the method defined in the Japanese Pharmacopoeia (fourteenth edition). The preservative effectiveness of respective test solutions was investigated for comparison.

Specifically, Pseudomonas aeruginosa ATCC 9027 strain was inoculated to the surface of agar plates and cultured thereon. As the agar plates, soybean casein digest agar medium was used, and the culturing condition was 32° C. for 24 hours. The cultured bacteria were aseptically collected by a platinum loop and suspended in a sterilized physiological saline solution, thereby preparing a bacteria-suspended solution containing the viable bacteria in an amount of about 1×10⁸ cells/ml. The bacteria were inoculated in liquid medium using the bacteria-suspended solution as a bacterium inoculation solution. The liquid medium were cultured, and centrifuged to remove supernatant. The bacteria thus obtained as precipitate were washed with a sterilized physiological saline solution and introduced into the test solutions having ingredients as shown in Tables 1 and 2, to contain bacteria in an amount of 1.6×10⁶ cells/ml.

The bacteria-containing test solutions were stored for 48 hours at a temperature of 25° C., and the number of viable cells per ml was then measured. The preservative efficacy of the respective test solutions was examined by calculating the log reduction in the number of viable cells before and after the storage.

Tables 1 and 2 show the results.

(1) Mixtures of xanthine, buffer and EDTA salt

TABLE 1

| | Ex. | Comparative Ex. | | | Ex. | Comparative Ex. | | |
|---|---|---|---|---|---|---|---|---|
| Content (g/100 ml) | 1 | 1 | 2 | 3 | 2 | 4 | 5 | 6 |
| Disodium hydrogenphosphate | 1.0 | 1.0 | 1.0 | 1.0 | — | — | — | — |
| Sodium dihydrogenphosphate | 0.2 | 0.2 | 0.2 | 0.2 | — | — | — | — |
| Boric Acid | — | — | — | — | 1.0 | 1.0 | 1.0 | 1.0 |
| Borax | — | — | — | — | 0.2 | 0.2 | 0.2 | 0.2 |
| Sodium edetate | 0.05 | — | — | 0.05 | 0.05 | — | — | 0.05 |
| Anhydrous caffein | 1.0 | 1.0 | — | — | 1.0 | 1.0 | — | — |
| HCl or NaOH | * | * | * | * | * | * | * | * |
| Purified water | * | * | * | * | * | * | * | * |
| pH | 7.47 | 7.52 | 7.50 | 7.50 | 7.49 | 7.50 | 7.49 | 7.50 |
| 48 h log reduction | 4.84 | 2.37 | 0 | 0.37 | 3.91 | 0.85 | 0.06 | 1.80 |

In Table 1, “*” shows “appropriate amount” and “Ex.” shows “Example”.

The results shown above reveal that the use of an EDTA (sodium edetate) combined with a xanthine (anhydrous caffeine) and a buffer (a borate buffer or phosphate buffer) result in preservative effectiveness more enhanced than that provided by compositions containing an EDTA (sodium edetate) and a buffer (a borate buffer or phosphate buffer), a xanthine (anhydrous caffeine) and a buffer (a borate buffer or phosphate buffer), or a buffer (a borate buffer or phosphate buffer).

(2) Mixtures of xanthine, buffer and sorbate, and mixtures of xanthine, buffer, sorbate and EDTA salt

TABLE 2

| | | | Comparative | | | | Comparative | |
|---|---|---|---|---|---|---|---|---|
| | Example | | Example | | Example | | Example | |
| Content (g/100 ml) | 3 | 4 | 7 | 8 | 5 | 6 | 9 | 10 |
| Disodium hydrogenphosphate | 1.0 | 1.0 | 1.0 | 1.0 | — | — | — | — |
| Sodium dihydrogenphosphate | 0.2 | 0.2 | 0.2 | 0.2 | — | — | — | — |
| Boric acid | — | — | — | — | 1.0 | 1.0 | 1.0 | 1.0 |
| Borax | — | — | — | — | 0.2 | 0.2 | 0.2 | 0.2 |
| Potassium sorbate | 0.1 | 0.1 | 0.1 | 0.1 | 0.1 | 0.1 | 0.1 | 0.1 |
| Sodium edetate | — | 0.05 | — | 0.05 | — | 0.05 | — | 0.05 |
| Anhydrous caffein | 1.0 | 1.0 | — | — | 1.0 | 1.0 | — | — |
| HCl or NaOH | * | * | * | * | * | * | * | * |
| Purified water | * | * | * | * | * | * | * | * |
| pH | 7.50 | 7.48 | 7.50 | 7.50 | 7.46 | 7.48 | 7.50 | 7.50 |
| 48 h log reduction | 3.12 | 6.20 | 0.06 | 0.52 | 2.18 | 5.00 | 0.36 | 2.18 |

In Table 2, “*” shows “appropriate amount”.

The results shown above reveal that the preservative effectiveness of the composition containing a sorbate combined with a xanthine (anhydrous caffeine) and a buffer (a borate buffer or phosphate buffer) is more enhanced than that of compositions containing a sorbate and a buffer (a borate buffer or phosphate buffer); and the addition of an EDTA (sodium edetate) to the above composition containing three ingredients further enhances the preservative effectiveness.

### Example 7

Preservative Composition

| | | |
|---|---|---|
| Caffeine-sodium benzoate | 5.0 | g |
| Sodium edetate | 0.5 | g |
| Potassium Sorbate | 3.0 | g |
| Sodium dihydrogenphosphate | 2.0 | g |
| Disodium hydrogenphosphate | 12.0 | g |

| | |
|---|---|
| Purified water | Balanced amount |

| | | |
|---|---|---|
| Total | 100 | ml |

### Example 8

Preservative Composition

| Caffeine-sodium benzoate | 1.0 | g |
|---|---|---|
| Caffeine | 0.5 | g |
| Sodium edetate | 0.05 | g |
| Potassium sorbate | 0.05 | g |
| Sodium dihydrogenphosphate | 0.10 | g |
| Disodium hydrogenphosphate | 0.60 | g |
| Purified water | Balanced amount | |
| Total | 100 | ml |

### Examples 9 to 55

Using the formulations shown in Tables 3 to 10, mixtures were prepared according to a known method. The mixtures were subjected to filter sterilization treatment and aseptically inserted into a vessel, thereby preparing ophthalmic formulations, collyriums and contact lens-care solutions.

TABLE 3

| | Ex. 9 | Ex. 10 | Ex. 11 | Ex. 12 | Ex. 13 |
|---|---|---|---|---|---|
| | Ophthalmic | Ophthalmic | Ophthalmic | Ophthalmic | Ophthalmic |
| Content (g/100 ml) | formulation | formulation | formulation | formulation | formulation |
| Tetrahydrozoline hydrochloride | — | — | 0.01 | 0.05 | 0.04 |
| Naphazoline hydrochloride | — | 0.003 | — | — | — |
| Neostigmine methylsulfate | — | 0.003 | — | 0.005 | — |
| ε-Aminocaproic acid | 1.0 | — | — | — | — |
| Allantoin | — | 0.06 | — | 0.20 | — |
| Dipotassium glycyrrhizinate | — | — | 0.25 | — | — |
| Chlorpheniramine maleate | 0.010 | 0.010 | 0.030 | 0.030 | 0.010 |
| Pyridoxine hydrochloride | — | 0.10 | — | 0.10 | 0.05 |
| Potassium L-aspartate | 0.2 | 1.0 | — | 1.0 | 1.0 |
| Aminoethylsulfonic acid | 0.500 | — | — | — | — |
| Sodium chondroitin sulfate | — | — | — | — | 0.5 |
| Boric acid | 1.00 | 1.00 | 1.80 | 1.00 | 1.00 |
| Borax | 0.20 | 0.09 | 0.05 | 0.05 | 0.04 |
| Hydrochloric acid | * | * | * | * | * |
| Sodium hydroxide | * | * | * | * | * |
| Sodium chloride | * | * | * | * | * |
| l-Menthol | 0.001 | 0.005 | 0.006 | 0.040 | 0.020 |
| d-Camphor | — | 0.010 | — | 0.005 | 0.003 |
| d-Borneol | — | 0.010 | 0.003 | — | — |
| Potassium sorbate | 0.10 | — | — | — | — |
| Chlorobutanol | — | 0.10 | 0.15 | — | 0.15 |
| Sodium edetate | — | 0.050 | 0.010 | 0.050 | 0.010 |
| Anhydrous caffeine | 0.1 | 1.0 | 0.6 | 1.0 | 0.4 |
| POE 60 Hydrogenated castor oil | 0.100 | — | — | 0.200 | — |
| Polysorbate 80 | — | 0.20 | 0.05 | — | 0.10 |
| Purified water | * | * | * | * | * |
| pH (20° C.) | 7.5 | 6.0 | 6.0 | 5.5 | 5.6 |
| Ratio of osmotic pressure | 1.2 | 1.3 | 1.1 | 1.3 | 1.2 |

* Appropriate amount

TABLE 4

| | Ex. 14 | Ex. 15 | Ex. 16 | Ex. 17 | Ex. 18 | Ex. 19 | Ex. 20 |
|---|---|---|---|---|---|---|---|
| | Ophthalmic | Ophthalmic | Ophthalmic | Ophthalmic | | | |
| Content (g/100 ml) | formulation | formulation | formulation | formulation | Collyrium | Collyrium | Collyrium |
| Neostigmine methylsulfate | — | 0.005 | 0.005 | — | — | — | — |
| ε-Aminocaproic acid | 2.0 | — | — | — | — | — | — |
| Dipotassium glycyrrhizinate | 0.10 | — | — | 0.25 | 0.03 | 0.03 | 0.03 |
| Chlorpheniramine maleate | 0.020 | 0.006 | 0.030 | 0.010 | 0.003 | 0.003 | 0.001 |
| Retinal palmitate | — | 25,000 U | — | — | — | — | — |
| dl-α-Tocopherol acetate | — | 0.05 | 0.05 | 0.03 | — | — | — |
| Potassium L-aspartate | — | 1.0 | 1.0 | 1.0 | 0.1 | 0.1 | 0.1 |
| Aminoethylsulfonic acid | — | 0.100 | — | 0.500 | — | 0.100 | — |
| Sodium chondroitin sulfate | — | — | 0.1 | — | — | — | — |
| Sulfamethoxazole sodium | 4.000 | — | — | — | — | — | — |
| Boric acid | — | — | — | — | 1.50 | — | 1.50 |
| Borax | — | — | — | — | 0.30 | — | 0.30 |
| Sodium dihydrogenphosphate | 0.20 | 0.10 | 0.10 | 0.10 | — | 0.20 | — |
| Disodium hydrogenphosphate | 1.20 | 0.60 | 0.60 | 0.60 | — | 1.20 | — |
| Hydrochloric acid | * | * | * | * | * | * | * |
| Sodium hydroxide | * | * | * | * | * | * | * |
| Sodium chloride | * | * | * | * | * | * | * |
| l-Menthol | — | 0.003 | 0.030 | 0.010 | 0.005 | 0.005 | 0.006 |
| d-Camphor | — | 0.002 | — | 0.003 | — | — | 0.006 |
| d-Borneol | — | — | 0.005 | — | — | — | 0.003 |
| Potassium sorbate | 0.05 | 0.10 | 0.05 | 0.10 | 0.10 | — | 0.01 |
| Sodium edetate | 0.010 | 0.005 | 0.010 | 0.010 | — | 0.050 | 0.010 |
| Anhydrous caffeine | 1.0 | 0.5 | 1.0 | 0.1 | 1.0 | 0.5 | 0.1 |
| Polysorbate 80 | 0.05 | 0.30 | 0.30 | 0.30 | 0.05 | 0.05 | 0.05 |
| Purified water | * | * | * | * | * | * | * |
| pH (20° C.) | 7.5 | 7.5 | 7.5 | 7.5 | 7.5 | 7.2 | 7.1 |
| Ratio of osmotic pressure | 1.1 | 1.3 | 1.1 | 1.2 | 1.2 | 1.2 | 1.2 |

* Appropriate amount

TABLE 5

| | Ex. 21 | Ex. 22 | Ex. 23 | Ex. 24 |
|---|---|---|---|---|
| | Ophthal- | Ophthal- | Ophthal- | Ophthal- |
| | mic for- | mic for- | mic for- | mic for- |
| Content (g/100 ml) | mulation | mulation | mulation | mulation |
| ε-Aminocaproic acid | 1.0 | — | — | — |
| Sodium azulene sulfonate | — | — | — | 0.002 |
| Dipotassium glycyrrhizi- | — | 0.1 | 0.2 | — |
| nate | | | | |
| Chlorpheniramine | 0.010 | — | 0.030 | — |
| maleate | | | | |
| Pyridoxine hydrochloride | 0.1 | 0.1 | — | — |
| Potassium L-aspartate | 0.2 | 0.2 | 1.0 | — |
| Aminoethylsulfonic acid | 0.1 | — | — | — |
| Sodium chondroitin sul- | — | 0.5 | — | — |
| fate | | | | |
| Pottasium chloride | 0.08 | — | 0.05 | 0.10 |
| Sodium chloride | * | * | * | * |
| Sodium hydrogencarbon- | 0.20 | — | — | — |
| ate | | | | |
| Sodium carbonate | 0.10 | — | — | — |
| Sodium citrate | — | — | 0.30 | — |
| Citric acid | — | — | 0.10 | — |
| Hydroxyethyl cellulose | — | — | — | — |
| Hydroxypropylmethyl | — | — | — | 0.20 |
| cellulose | | | | |
| Glucose | — | — | — | 0.05 |
| Boric acid | — | 1.0 | — | 0.5 |
| Borax | — | 0.20 | — | 0.02 |
| Hydrochloric acid | * | * | * | * |
| Sodium hydroxide | * | * | * | * |
| l-Menthol | 0.005 | 0.010 | — | — |
| d-Camphor | 0.002 | 0.001 | — | — |
| d-Borneol | — | 0.001 | — | — |
| Potassium sorbate | — | 0.1 | 0.1 | 0.1 |
| Sorbic acid | 0.1 | — | — | — |
| Sodium edetate | 0.050 | 0.005 | 0.100 | 0.003 |
| Ethylenediaminetetra- | — | — | — | 0.003 |
| acetic acid | | | | |
| Anhydrous caffeine | 0.1 | 0.5 | 0.3 | 0.3 |
| POE 60 hydrogenated | 0.10 | — | — | 0.10 |
| castor oil | | | | |
| Polysorbate 80 | 0.10 | 0.20 | 0.05 | — |
| Poloxamer 407 | — | — | 0.05 | 0.10 |
| Purified water | * | * | * | * |
| pH (20° C.) | 6.8 | 7.0 | 7.2 | 6.8 |
| Ratio of osmotic pressure | 1.4 | 1.1 | 1.1 | 1.1 |

*Appropriate amount

TABLE 6

| | Ex. 25 | Ex. 26 | Ex. 27 | Ex. 28 | Ex. 29 | Ex. 30 | Ex. 31 |
|---|---|---|---|---|---|---|---|
| | Ophthalmic | Ophthalmic | Ophthalmic | Ophthalmic | Ophthalmic | | |
| Content (g/100 ml) | formulation | formulation | formulation | formulation | formulation | Collyrium | Collyrium |
| Sodium chondroitin sulfate | — | — | — | 0.5 | 0.5 | — | — |
| Potassium chloride | 0.05 | 0.05 | 0.10 | 0.05 | 0.10 | 0.15 | 0.10 |
| Calcium chloride | — | — | — | — | — | — | 0.050 |
| Sodium chloride | * | * | * | * | * | * | * |
| Sodium hydrogencarbonate | — | — | — | — | — | — | 0.05 |
| Disodium hydrogenphosphate | — | — | 0.20 | 0.20 | — | — | 0.20 |
| Potassium dihydrogenphosphate | — | — | 0.05 | 0.05 | — | — | 0.05 |
| Polyvinyl alcohol | — | — | — | 1.000 | — | — | — |
| Polyvinyl pyrrolidone | — | — | — | — | 0.05 | — | — |
| Hydroxyethyl cellulose | 0.03 | 0.03 | — | — | — | — | 0.10 |
| Hydroxypropylmethyl cellulose | — | — | — | — | — | 0.10 | — |
| Boric acid | 1.0 | 1.0 | — | — | 1.0 | 0.3 | — |
| Borax | 0.15 | 0.15 | — | — | 0.15 | 0.02 | — |
| Hydrochloric acid | * | * | * | * | * | * | * |
| Sodium hydroxide | * | * | * | * | * | * | * |
| l-Menthol | 0.002 | 0.001 | — | — | — | 0.002 | — |
| d-Camphor | 0.001 | — | 0.002 | — | — | — | — |
| d-Borneol | 0.001 | — | 0.002 | — | — | 0.001 | — |
| Potassium sorbate | — | 0.1 | — | 0.1 | 0.1 | 0.1 | 0.1 |
| Sorbic acid | 0.2 | — | 0.1 | — | — | — | — |
| Sodium edetate | 0.050 | 0.050 | — | 0.010 | — | 0.003 | 0.003 |
| Ethylenediaminetetraacetic acid | — | — | 0.003 | — | 0.003 | — | 0.003 |
| Anhydrous caffeine | 0.3 | 0.3 | 1.0 | 0.3 | 1.0 | 0.2 | 0.2 |
| POE 60 hydrogenated castor oil | 0.05 | 0.05 | — | 0.05 | 0.03 | 0.05 | 0.05 |
| Polysorbate 80 | — | — | 0.05 | 0.05 | 0.03 | — | — |
| Poloxamer 407 | 0.05 | 0.05 | 0.05 | 0.05 | 0.03 | 0.05 | 0.05 |
| Purified water | * | * | * | * | * | * | * |
| pH (20° C.) | 7.0 | 7.2 | 6.8 | 7.0 | 7.2 | 6.8 | 7.0 |
| Ratio of osmotic pressure | 1.0 | 1.0 | 1.1 | 1.2 | 1.0 | 1.0 | 1.2 |

* Appropriate amount

TABLE 7

| | | Ex. 33 | Ex. 34 | Ex. 35 |
|---|---|---|---|---|
| | | Contact | Contact | Contact |
| | | lens- | lens- | lens- |
| | Ex. 32 | care | care | care |
| Content (g/100 ml) | Collyrium | solution | solution | solution |
| Dipotassium glycyrrhizinate | 0.1 | — | 0.2 | — |
| Sodium chondroitin sulfate | — | — | — | 0.1 |
| Potassium chloride | 0.10 | 0.10 | 0.05 | — |
| Sodium chloride | * | * | * | * |
| Disodium hydrogenphosphate | — | 0.20 | — | — |
| Potassium dihydrogenphosphate | — | 0.05 | — | — |
| Sodium citrate | 0.30 | — | 0.30 | — |
| Citric acid | 0.10 | — | 0.10 | — |
| Hydroxypropylmethyl cellulose | — | 0.05 | — | — |
| Sodium alginate | 0.1 | — | — | 0.1 |
| Glycerin | — | 0.1 | — | — |
| Boric acid | — | — | — | 0.5 |
| Borax | — | — | — | 0.10 |
| Hydrochloric acid | * | * | * | * |
| Sodium hydroxide | * | * | * | * |
| l-Menthol | 0.005 | 0.002 | — | — |
| Potassium sorbate | — | — | 0.1 | — |
| Sorbic acid | 0.2 | 0.1 | — | 0.2 |
| Sodium edetate | 0.010 | — | 0.050 | 0.050 |
| Ethylenediaminetetraacetic acid | — | — | 0.003 | — |
| Anhydrous caffeine | 0.8 | 0.5 | 0.5 | 0.1 |
| POE 60 hydrogenated castor oil | — | — | — | 0.05 |
| Polysorbate 80 | 0.10 | 0.01 | — | — |
| Poloxamer 407 | — | 0.10 | 0.10 | 0.05 |
| Purified water | * | * | * | * |
| pH (20° C.) | 7.2 | 6.8 | 7.0 | 7.2 |
| Ratio of osmotic pressure | 1.1 | 1.0 | 1.2 | 1.1 |

*Appropriate amount

TABLE 8

| | Ex. 36 | Ex. 37 | Ex. 38 | Ex. 39 | Ex. 40 | Ex. 41 | Ex. 42 |
|---|---|---|---|---|---|---|---|
| | Ophthalmic | Ophthalmic | Ophthalmic | Ophthalmic | Ophthalmic | Ophthalmic | Ophthalmic |
| Content (g/100 ml) | formulation | formulation | formulation | formulation | formulation | formulation | formulation |
| Neostigmine methylsulfate | — | 0.002 | — | 0.005 | — | — | 0.005 |
| Tetrahydrozoline hydrochloride | 0.02 | — | — | 0.02 | 0.03 | 0.05 | — |
| ε-Aminocaproic acid | — | — | 1.0 | — | — | — | — |
| Allantoin | — | 0.1 | — | 0.1 | — | — | — |
| Sodium azulene sulfonate | 0.02 | — | — | — | — | — | — |
| Dipotassium glycyrrhizinate | — | 0.10 | 0.10 | — | 0.25 | — | — |
| Zinc sulfate | — | 0.1 | — | — | — | — | — |
| Chlorpheniramine maleate | 0.03 | 0.03 | 0.01 | 0.01 | 0.03 | 0.01 | 0.03 |
| Pyridoxine hydrochloride | — | — | — | 0.10 | — | 0.05 | 0.10 |
| Cyanocobalamin | — | — | — | — | — | 0.006 | — |
| d-α-Tocopherol acetate | — | — | — | — | — | — | 0.05 |
| Potassium L-aspartate | — | — | 1.0 | 1.0 | — | 1.0 | 1.0 |
| Aminoethylsulfonic acid | — | — | — | — | — | 0.5 | — |
| Hydroxypropylmethyl cellulose | — | — | — | — | — | 0.1 | — |
| Boric acid | 1.80 | 1.80 | 1.00 | 1.00 | 1.80 | 1.00 | 1.00 |
| Borax | 0.30 | 0.01 | 0.20 | 0.05 | 0.05 | 0.05 | 0.10 |
| Chlorobutanol | — | — | — | — | — | 0.15 | 0.25 |
| l-Menthol | 0.010 | 0.005 | 0.002 | 0.010 | 0.005 | 0.020 | 0.020 |
| d-Camphor | 0.005 | 0.002 | 0.001 | 0.005 | 0.003 | 0.005 | 0.005 |
| d-Borneol | 0.002 | 0.001 | 0.001 | — | — | 0.001 | 0.002 |
| Sorbic acid | — | — | — | — | — | — | 0.1 |
| Sodium edetate | 0.005 | 0.010 | 0.010 | 0.010 | 0.050 | 0.010 | — |
| Anhydrous caffeine | 1.0 | 0.5 | 0.5 | 0.5 | 0.8 | 0.5 | 0.2 |
| POE 60 hydrogenated castor oil | — | — | — | 0.2 | — | — | — |
| Polysorbate 80 | 0.2 | 0.1 | 0.1 | — | 0.1 | 0.1 | 0.3 |
| Hydrochloric acid | * | * | * | * | * | * | * |
| Sodium hydroxide | * | * | * | * | * | * | * |
| Purified water | * | * | * | * | * | * | * |
| pH (20° C.) | 7.2 | 5.0 | 7.5 | 5.2 | 5.5 | 6.0 | 5.5 |
| Ratio of osmotic pressure | 1.1 | 1.1 | 1.2 | 1.2 | 1.2 | 1.2 | 1.3 |

* Appropriate amount

TABLE 9

| | Ex. 43 | Ex. 44 | Ex. 45 | Ex. 46 | Ex. 47 | Ex. 48 |
|---|---|---|---|---|---|---|
| | Ophthalmic | Ophthalmic | Ophthalmic | Ophthalmic | Ophthalmic | Ophthalmic |
| Content (g/100 ml) | formulation | formulation | formulation | formulation | formulation | formulation |
| Neostigmine methylsulfate | 0.005 | — | — | — | — | — |
| Tetrahydrozoline hydrochloride | — | 0.01 | — | — | — | — |
| ε-Aminocaproic acid | — | 0.5 | 0.5 | — | — | — |
| Dipotassium glycyrrhizinate | — | — | 0.15 | — | — | — |
| Zinc sulfate | — | — | — | — | — | |
| Chlorpheniramine maleate | 0.01 | — | 0.03 | — | — | — |
| Retinol palmitate | 50,000 U | — | — | — | — | — |
| Pyridoxine hydrochloride | — | 0.05 | — | — | — | — |
| d-α-Tocopherol acetate | 0.05 | — | — | — | — | — |
| Panthenol | — | 0.1 | — | — | — | — |
| Potassium L-aspartate | 1.0 | 1.0 | — | — | — | — |
| Sodium chondroitin sulfate | — | — | — | 0.5 | — | — |
| Sulfamethoxazole sodium | — | — | 4.0 | — | — | — |
| Potassium chloride | — | — | — | 0.1 | 0.1 | 0.1 |
| Sodium chloride | — | 0.3 | — | 0.5 | 0.5 | 0.5 |
| Sodium hydrogencarbonate | 0.05 | — | — | 0.05 | — | — |
| Hydroxyethyl cellulose | — | — | — | 0.02 | — | 0.05 |
| Hydroxypropylmethyl cellulose | 0.20 | — | 0.10 | — | 0.25 | — |
| Glucose | — | — | — | — | 0.09 | — |
| Boric acid | 1.00 | — | 0.20 | 1.00 | 1.00 | 1.00 |
| Borax | 0.10 | — | 0.20 | 0.20 | 0.15 | 0.20 |
| Chlorobutanol | 0.10 | 0.15 | — | — | — | — |
| l-Menthol | 0.010 | 0.010 | 0.020 | 0.002 | 0.003 | 0.005 |
| d-Camphor | — | 0.002 | 0.005 | — | 0.001 | — |
| d-Borneol | 0.002 | — | — | — | — | — |
| Potassium sorbate | — | 0.1 | — | — | — | — |
| Sodium edetate | 0.005 | — | 0.005 | 0.050 | 0.050 | 0.003 |
| Anhydrous caffeine | 0.5 | 1.0 | 0.5 | 0.5 | 0.5 | 0.5 |
| Polysorbate 80 | 0.25 | 0.20 | 0.10 | 0.05 | 0.05 | — |
| Poloxamer 407 | — | — | — | 0.05 | 0.05 | 0.05 |
| Propylene glycol | — | — | — | 0.25 | 0.25 | 0.25 |
| Hydrochloric acid | * | * | * | * | * | * |
| Sodium hydroxide | * | * | * | * | * | * |
| Purified water | * | * | * | * | * | * |
| pH (20° C.) | 6.0 | 5.5 | 8.5 | 7.4 | 7.0 | 7.5 |
| Ratio of osmotic pressure | 1.2 | 1.2 | 1.3 | 1.3 | 1.3 | 1.3 |

* Appropriate amount

TABLE 10

| | Ex. 49 | Ex. 50 | | | | | |
|---|---|---|---|---|---|---|---|
| | Ophthalmic | Ophthalmic | Ex. 51 | Ex. 52 | Ex. 53 | Ex. 54 | Ex. 55 |
| Content (g/100 ml) | formulation | formulation | Collyrium | Collyrium | Collyrium | Collyrium | ** |
| ε-Aminocaproic acid | — | — | — | — | 0.025 | — | — |
| Dipotassium glycyrrhizinate | 0.125 | 0.050 | — | 0.025 | 0.025 | 0.010 | — |
| Zinc sulfate | — | 0.050 | 0.025 | — | 0.010 | — | — |
| Chlorpheniramine maleate | — | — | — | 0.0030 | 0.0030 | 0.0006 | — |
| Pyridoxine hydrochloride | 0.01 | — | — | — | — | 0.01 | — |
| d-α-Tocopherol acetate | — | — | — | — | — | 0.005 | — |
| Potassium L-aspartate | — | — | — | 0.10 | 0.10 | 0.05 | — |
| Aminoethyl sulfonate | — | — | — | — | — | 0.05 | — |
| Sodium chondroitin sulfate | — | — | — | — | — | — | 0.1 |
| Potassium chloride | — | — | — | — | — | — | 0.25 |
| Sodium chloride | 0.70 | 0.35 | 0.50 | — | — | — | 0.80 |
| Polyvinyl pyrrolidone | — | 0.10 | — | — | — | — | 1.25 |
| Hydroxypropylmethyl cellulose | — | — | 0.05 | — | — | — | — |
| Boric acid | 0.700 | 1.000 | 1.000 | 1.800 | 1.800 | 1.500 | 0.150 |
| Borax | 0.100 | 0.025 | 0.010 | 0.100 | 0.015 | 0.300 | 0.020 |
| Chlorobutanol | — | — | — | — | 0.05 | — | — |
| l-Menthol | 0.010 | 0.005 | 0.002 | 0.005 | 0.010 | 0.005 | — |
| d-Camphor | 0.005 | — | — | 0.005 | 0.005 | — | — |
| dl-Camphor | — | — | — | — | — | 0.050 | — |
| d-Borneol | — | — | — | 0.002 | 0.001 | — | — |
| Sorbic acid | — | — | 0.1 | — | — | — | — |
| Sodium edetate | 0.004 | 0.002 | — | 0.005 | 0.005 | 0.005 | 0.025 |
| Anhydrous caffeine | 0.5 | 0.1 | 0.1 | 0.5 | 0.3 | 0.5 | 1.0 |
| Polysorbate 80 | — | 0.05 | 0.05 | 0.05 | 0.05 | 0.10 | 0.01 |
| Poloxamer 407 | 0.05 | — | — | — | — | — | 0.01 |
| Propylene glycol | — | 0.5 | — | — | — | — | — |
| Hydrochloric acid | * | * | * | * | * | * | * |
| Sodium hydroxide | * | * | * | * | * | * | * |
| Purified water | * | * | * | * | * | * | * |
| pH (20° C.) | 7.2 | 5.5 | 5.0 | 6.5 | 6.0 | 7.2 | 7.5 |
| Ratio of osmotic pressure | 1.2 | 1.2 | 1.3 | 1.2 | 1.2 | 1.2 | 1.4 |

* Appropriate amount
** Contact lens-wearing solution

Industrial Applicability

The composition of the present invention comprises in combination (a) a xanthine, (b) a buffer and (c) at least one member selected from sorbic acid, EDTA, and salts thereof, thereby exhibiting safe and superior preservative properties. In a variety of fields such as pharmaceuticals, quasi-medical products, cosmetics and articles for daily use (miscellaneous articles), the composition of the present invention is useful as a composition that is safe and of superior preservative efficacy, and especially useful as a composition that is suitable for application to the skin and mucous membranes.

When the composition of the present invention contains sorbic acid or a salt thereof, the use of a xanthine moderates the irritation to the skin and mucous membranes caused by the sorbic acid or a salt thereof contained in the composition. Therefore, although the composition of the present invention contains sorbic acid or a salt thereof, it does not exhibit irritation to the skin or especially mucous membranes and is very safe, and thus suitable for use as ophthalmic compositions such as ophthalmic solutions (including those usable while contact lenses are in use), ophthalmic ointment, contact lens-wearing solutions, collyriums (including contact lens-friendly collyriums usable while contact lenses are in use), contact lens-care formulations (cleaning solutions, soaking solutions, disinfecting solutions, multi-purpose solutions); ear and nasal compositions such as nasal drops, ear drops, nasal washing fluid; and oral compositions such as oropharyngeal formulations, mouthwash, etc.

Furthermore, according to the method of the present invention for enhancing preservative properties, the preservative properties of sorbic acid, EDTA and its salts, and a xanthine, which are known to have preservative properties, can be enhanced. Thereby, the amount of sorbic acid, EDTA, and salts thereof used can be reduced, and preservative efficacy can be more safely imparted to pharmaceuticals, quasi-medical products, cosmetics, food products, articles for daily use (miscellaneous articles) and various like products.

1. A composition comprising:
(a) a compound represented by Formula (I): 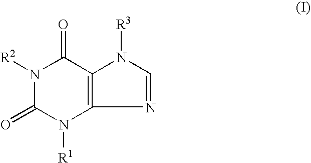
wherein R¹, R² and R³ are the same or different, and individually represent hydrogen or optionally substituted alkyl, or a salt of the compound;
(b) a buffer; and
(c) at least one member selected from sorbic acid, ethylenediaminetetraacetic acid, and salts thereof:

2. A composition according to claim 1, wherein the composition is an aqueous composition.

3. A composition according to claim 1, wherein the composition is suitable for application to the skin or mucous membranes.

4. A composition according to claim 1, wherein the composition is an ophthalmic composition or an oral composition.

5. A composition according to claim 1, wherein the buffer is a borate buffer or phosphate buffer.

6. A composition according to claim 1, wherein Compound (I) is at least one compound selected from caffeine, oxtriphylline, dyphylline, diisobutylaminobenzoyloxypropyl theophylline, pentoxifylline, theophylline, diprophylline, theobromine and proxyphylline.

7. A composition according to claim 1, wherein Compound (I) is at least one compound selected from caffeine, pentoxifylline, theophylline, diprophylline, theobromine and proxyphylline.

8. A composition according to claim 1, wherein Compound (I) is at least one compound selected from caffeine-sodium benzoate, caffeine citrate, aminophylline, ambuphylline, theobromine calcium salicylate, theobromine sodium salicylate and theobromine sodium acetate.

9. A composition according to claim 1 containing (a) Compound (I) or a salt thereof in a proportion of 0.0001 to 10 w/v %, (b) the buffer in a proportion of 0.0001 to 10 w/v %, and (c) the sorbic acid or a salt thereof in a proportion of 0.00005 to 10 w/v %, or the ethylenediaminetetraacetic acid or a salt thereof in a proportion of 0.001 to 1 w/v %.

10. A composition according to claim 1, wherein the composition contains (b) the buffer in a proportion of 0.1 to 500 parts by weight and (c) the sorbic acid or a salt thereof in a total proportion of 0.002 to 100 parts by weight, or the ethylenediaminetetraacetic acid or a salt thereof in a total proportion of 0.001 to 1,000 parts by weight per part by weight of the total amount of (a) Compound (I) or a salt thereof.

11. A composition according to claim 1, wherein the composition is an ophthalmic solution, collyrium, ophthalmic ointment, contact lens-wearing solution or contact lens-care formulation.

12. A method for synergistically enhancing the preservative properties of the following ingredient (a) or (c), the method comprising employing in combination (a) a compound represented by Formula (I): 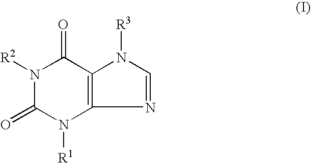
wherein R¹, R² and R³ are the same or different, and individually represent hydrogen or optionally substituted alkyl, or a salt of the compound;
(b) a buffer; and
(c) at least one member selected from sorbic acid, ethylenediaminetetraacetic acid, and salts thereof.

13. A method for enhancing the preservative properties of the following ingredient (c), the method comprising employing
(a) a compound represented by following Formula (I): 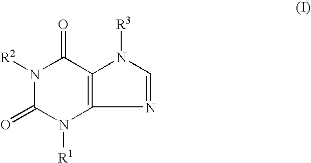
wherein R¹, R² and R³ are the same or different, and individually represent hydrogen or optionally substituted alkyl, or a salt of the compound, and
(b) a buffer in combination with
(c) at least one member selected from sorbic acid, ethylenediaminetetraacetic acid and salts thereof.

14. A method for enhancing the preservative properties of a composition comprising (b) a buffer and (c) at least one member selected from sorbic acid, ethylenediaminetetraacetic acid, and salts thereof, the method comprising employing the composition in combination with (a) a compound represented by Formula (I): 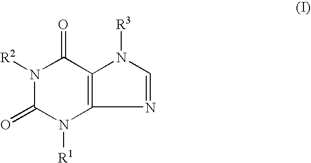
wherein R¹, R² and R³ are the same or different, and individually represent hydrogen or optionally substituted alkyl, or a salt of the compound.

15. A method for enhancing the preservative properties of a compound (I) represented by Formula (I): 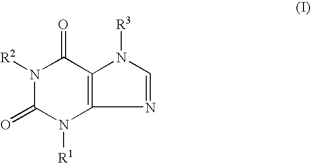
wherein R¹, R² and R³ are the same or different, and individually represent hydrogen or optionally substituted alkyl, or a salt of the compound, the method comprising employing:
(a) the Compound (I) above or a salt thereof, in combination with
(b) a buffer and
(c) at least one member selected from sorbic acid, ethylenediaminetetraacetic acid, and salts thereof.

16. A method for enhancing the preservative properties of a composition containing
(a) a compound represented by Formula (I): 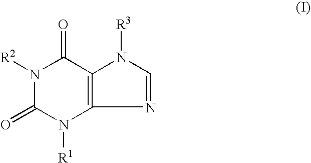
wherein R¹, R² and R³ are the same or different, and individually represent hydrogen or optionally substituted alkyl, or a salt of the compound and
(c) at least one member selected from sorbic acid, ethylenediaminetetraacetic acid, and salts thereof, comprising employing the composition in combination with
(b) a buffer.
